SEQUENCE LISTING (42 sequences)

SEQ ID NO: 1 - protein, 267 aa, Human
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 2 - protein, 250 aa, Human
MAHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 3 - protein, 100 aa, Human
MKLLAATVLLLTICSLEGALVRRQAKEPCVESLVSQYFQTVTDYGKDLMEKVKSPELQAEAKSYFEKSKEQLTPLIKKAGTELVNFLSYFVELGTQPATQ

SEQ ID NO: 4 - protein, 396 aa, Human
MFLKAVVLTLALVAVAGARAEVSADQVATVMWDYFSQLSNNAKEAVEHLQKSELTQQLNALFQDKLGEVNTYAGDLQKKLVPFATELHERLAKDSEKLKEEIGKELEELRARLLPHANEVSQKIGDNLRELQQRLEPYADQLRTQVNTQAEQLRRQLDPLAQRMERVLRENADSLQASLRPHADELKAKIDQNVEELKGRLTPYADEFKVKIDQTVEELRRSLAPYAQDTQEKLNHQLEGLTFQMKKNAEELKARISASAEELRQRLAPLAEDVRGNLKGNTEGLQKSLAELGGHLDQQVEEFRRRVEPYGENFNKALVQQMEQLRQKLGPHAGDVEGHLSFLEKDLRDKVNSFFSTFKEKESQDKTLSLPELEQQQEQQQEQQQEQVQMLAPLES

SEQ ID NO: 5 - protein, 366 aa, Human
MASMAAVLTWALALLSAFSATQARKGFWDYFSQTSGDKGRVEQIHQQKMAREPATLKDSLEQDLNNMNKFLEKLRPLSGSEAPRLPQDPVGMRRQLQEELEEVKARLQPYMAEAHELVGWNLEGLRQQLKPYTMDLMEQVALRVQELQEQLRVVGEDTKAQLLGGVDEAWALLQGLQSRVVHHTGRFKELFHPYAESLVSGIGRHVQELHRSVAPHAPASPARLSRCVQVLSRKLTLKAKALHARIQQNLDQLREELSRAFAGTGTEEGAGPDPQMLSEEVRQRLQAFRQDTYLQIAAFTRAIDQETEEVQQQLAPPPPGHSAFAPEFQQTDSGKVLSKLQARLDDLWEDITHSLHDQGHSHLGDP

SEQ ID NO: 6 - protein, 4563 aa, Human
MDPPRPALLALLALPALLLLLLAGARAEEEMLENVSLVCPKDATRFKHLRKYTYNYEAESSSGVPGTADSRSATRINCKVELEVPQLCSFILKTSQCTLKEVYGFNPEGKALLKKTKNSEEFAAAMSRYELKLAIPEGKQVFLYPEKDEPTYILNIKRGIISALLVPPETEEAKQVLFLDTVYGNCSTHFTVKTRKGNVATEISTERDLGQCDRFKPIRTGISPLALIKGMTRPLSTLISSSQSCQYTLDAKRKHVAEAICKEQHLFLPFSYNNKYGMVAQVTQTLKLEDTPKINSRFFGEGTKKMGLAFESTKSTSPPKQAEAVLKTLQELKKLTISEQNIQRANLFNKLVTELRGLSDEAVTSLLPQLIEVSSPITLQALVQCGQPQCSTHILQWLKRVHANPLLIDVVTYLVALIPEPSAQQLREIFNMARDQRSRATLYALSHAVNNYHKTNPTGTQELLDIANYLMEQIQDDCTGDEDYTYLILRVIGNMGQTMEQLTPELKSSILKCVQSTKPSLMIQKAAIQALRKMEPKDKDQEVLLQTFLDDASPGDKRLAAYLMLMRSPSQADINKIVQILPWEQNEQVKNFVASHIANILNSEELDIQDLKKLVKEALKESQLPTVMDFRKFSRNYQLYKSVSLPSLDPASAKIEGNLIFDPNNYLPKESMLKTTLTAFGFASADLIEIGLEGKGFEPTLEALFGKQGFFPDSVNKALYWVNGQVPDGVSKVLVDHFGYTKDDKHEQDMVNGIMLSVEKLIKDLKSKEVPEARAYLRILGEELGFASLHDLQLLGKLLLMGARTLQGIPQMIGEVIRKGSKNDFFLHYIFMENAFELPTGAGLQLQISSSGVIAPGAKAGVKLEVANMQAELVAKPSVSVEFVTNMGIIIPDFARSGVQMNTNFFHESGLEAHVALKAGKLKFIIPSPKRPVKLLSGGNTLHLVSTTKTEVIPPLIENRQSWSVCKQVFPGLNYCTSGAYSNASSTDSASYYPLTGDTRLELELRPTGEIEQYSVSATYELQREDRALVDTLKFVTQAEGAKQTEATMTFKYNRQSMTLSSEVQIPDFDVDLGTILRVNDESTEGKTSYRLTLDIQNKKITEVALMGHLSCDTKEERKIKGVISIPRLQAEARSEILAHWSPAKLLLQMDSSATAYGSTVSKRVAWHYDEEKIEFEWNTGTNVDTKKMTSNFPVDLSDYPKSLHMYANRLLDHRVPETDMTFRHVGSKLIVAMSSWLQKASGSLPYTQTLQDHLNSLKEFNLQNMGLPDFHIPENLFLKSDGRVKYTLNKNSLKIEIPLPFGGKSSRDLKMLETVRTPALHFKSVGFHLPSREFQVPTFTIPKLYQLQVPLLGVLDLSTNVYSNLYNWSASYSGGNTSTDHFSLRARYHMKADSVVDLLSYNVQGSGETTYDHKNTFTLSCDGSLRHKFLDSNIKFSHVEKLGNNPVSKGLLIFDASSSWGPQMSASVHLDSKKKQHLFVKEVKIDGQFRVSSFYAKGTYGLSCQRDPNTGRLNGESNLRFNSSYLQGTNQITGRYEDGTLSLTSTSDLQSGIIKNTASLKYENYELTLKSDTNGKYKNFATSNKMDMTFSKQNALLRSEYQADYESLRFFSLLSGSLNSHGLELNADILGTDKINSGAHKATLRIGQDGISTSATTNLKCSLLVLENELNAELGLSGASMKLTTNGRFREHNAKFSLDGKAALTELSLGSAYQAMILGVDSKNIFNFKVSQEGLKLSNDMMGSYAEMKFDHTNSLNIAGLSLDFSSKLDNIYSSDKFYKQTVNLQLQPYSLVTTLNSDLKYNALDLTNNGKLRLEPLKLHVAGNLKGAYQNNEIKHIYAISSAALSASYKADTVAKVQGVEFSHRLNTDIAGLASAIDMSTNYNSDSLHFSNVFRSVMAPFTMTIDAHTNGNGKLALWGEHTGQLYSKFLLKAEPLAFTFSHDYKGSTSHHLVSRKSISAALEHKVSALLTPAEQTGTWKLKTQFNNNEYSQDLDAYNTKDKIGVELTGRTLADLTLLDSPIKVPLLLSEPINIIDALEMRDAVEKPQEFTIVAFVKYDKNQDVHSINLPFFETLQEYFERNRQTIIVVVENVQRNLKHINIDQFVRKYRAALGKLPQQANDYLNSFNWERQVSHAKEKLTALTKKYRITENDIQIALDDAKINFNEKLSQLQTYMIQFDQYIKDSYDLHDLKIAIANIIDEIIEKLKSLDEHYHIRVNLVKTIHDLHLFIENIDFNKSGSSTASWIQNVDTKYQIRIQIQEKLQQLKRHIQNIDIQHLAGKLKQHIEAIDVRVLLDQLGTTISFERINDVLEHVKHFVINLIGDFEVAEKINAFRAKVHELIERYEVDQQIQVLMDKLVELTHQYKLKETIQKLSNVLQQVKIKDYFEKLVGFIDDAVKKLNELSFKTFIEDVNKFLDMLIKKLKSFDYHQFVDETNDKIREVTQRLNGEIQALELPQKAEALKLFLEETKATVAVYLESLQDTKITLIINWLQEALSSASLAHMKAKFRETLEDTRDRMYQMDIQQELQRYLSLVGQVYSTLVTYISDWWTLAAKNLTDFAEQYSIQDWAKRMKALVEQGFTVPEIKTILGTMPAFEVSLQALQKATFQTPDFIVPLTDLRIPSVQINFKDLKNIKIPSRFSTPEFTILNTFHIPSFTIDFVEMKVKIIRTIDQMQNSELQWPVPDIYLRDLKVEDIPLARITLPDFRLPEIAIPEFIIPTLNLNDFQVPDLHIPEFQLPHISHTIEVPTFGKLYSILKIQSPLFTLDANADIGNGTTSANEAGIAASITAKGESKLEVLNFDFQANAQLSNPKINPLALKESVKFSSKYLRTEHGSEMLFFGNAIEGKSNTVASLHTEKNTLELSNGVIVKINNQLTLDSNTKYFHKLNIPKLDFSSQADLRNEIKTLLKAGHIAWTSSGKGSWKWACPRFSDEGTHESQISFTIEGPLTSFGLSNKINSKHLRVNQNLVYESGSLNFSKLEIQSQVDSQHVGHSVLTAKGMALFGEGKAEFTGRHDAHLNGKVIGTLKNSLFFSAQPFEITASTNNEGNLKVRFPLRLTGKIDFLNNYALFLSPSAQQASWQVSARFNQYKYNQNFSAGNNENIMEAHVGINGEANLDFLNIPLTIPEMRLPYTIITTPPLKDFSLWEKTGLKEFLKTTKQSFDLSVKAQYKKNKHRHSITNPLAVLCEFISQSIKSFDRHFEKNRNNALDFVTKSYNETKIKFDKYKAEKSHDELPRTFQIPGYTVPVVNVEVSPFTIEMSAFGYVFPKAVSMPSFSILGSDVRVPSYTLILPSLELPVLHVPRNLKLSLPHFKELCTISHIFIPAMGNITYDFSFKSSVITLNTNAELFNQSDIVAHLLSSSSSVIDALQYKLEGTTRLTRKRGLKLATALSLSNKFVEGSHNSTVSLTTKNMEVSVAKTTKAEIPILRMNFKQELNGNTKSKPTVSSSMEFKYDFNSSMLYSTAKGAVDHKLSLESLTSYFSIESSTKGDVKGSVLSREYSGTIASEANTYLNSKSTRSSVKLQGTSKIDDIWNLEVKENFAGEATLQRIYSLWEHSTKNHLQLEGLFFTNGEHTSKATLELSPWQMSALVQVHASQPSSFHDFPDLGQEVALNANTKNQKIRWKNEVRIHSGSFQSQVELSNDQEKAHLDIAGSLEGHLRFLKNIILPVYDKSLWDFLKLDVTTSIGRRQHLRVSTAFVYTKNPNGYSFSIPVKVLADKFITPGLKLNDLNSVLVMPTFHVPFTDLQVPSCKLDFREIQIYKKLRTSSFALNLPTLPEVKFPEVDVLTKYSQPEDSLIPFFEITVPESQLTVSQFTLPKSVSDGIAALDLNAVANKIADFELPTIIVPEQTIEIPSIKFSVPAGIVIPSFQALTARFEVDSPVYNATWSASLKNKADYVETVLDSTCSSTVQFLEYELNVLGTHKIEDGTLASKTKGTLAHRDFSAEYEEDGKFEGLQEWEGKAHLNIKSPAFTDLHLRYQKDKKGISTSAASPAVGTVGMDMDEDDDFSKWNFYYSPQSSPDKKLTIFKTELRVRESDEETQIKVNWEEEAASGLLTSLKDNVPKATGVLYDYVNKYHWEHTGLTLREVSSKLRRNLQNNAEWVYQGAIRQIDDIDVRFQKAASGTTGTYQEWKDKAQNLYQELLTQEGQASFQGLKDNVFDGLVRVTQKFHMKVKHLIDSLIDFLNFPRFQFPGKPGIYTREELCTMFIREVGTVLSQVYSKVHNGSEILFSYFQDLVITLPFELRKHKLIDVISMYRELLKDLSKEAQEVFKAIQSLKTTEVLRNLQDLLQFIFQLIEDNIKQLKEMKFTYLINYIQDEINTIFNDYIPYVFKLLKENLCLNLHKFNEFIQNELQEASQELQQIHQYIMALREEYFDPSIVGWTVKYYELEEKIVSLIKNLLVALKDFHSEYIVSASNFTSQLSSQVEQFLHRNIQEYLSILTDPDGKGKEKIAELSATAQEIIKSQAIATKKIISDYHQQFRYKLQDFSDQLSDYYEKFIAESKRLIDLSIQNYHTFLIYITELLKKLQSTTVMNPYMKLAPGELTIIL

SEQ ID NO: 7 - protein, 728 aa, Human
LNAELGLSGASMKLTTNGRFREHNAKFSLDGKAALTELSLGSAYQAMILGVDSKNIFNFKVSQEGLKLSNDMMGSYAEMKFDHTNSLNIAGLSLDFSSKLDNIYSSDKFYKQTVNLQLQPYSLVTTLNSDLKYNALDLTNNGKLRLEPLKLHVAGNLKGAYQNNEIKHIYAISSAALSASYKADTVAKVQGVEFSHRLNTDIAGLASAIDMSTNYNSDSLHFSNVFRSVMAPFTMTIDAHTNGNGKLALWGEHTGQLYSKFLLKAEPLAFTFSHDYKGSTSHHLVSRKSISAALEHKVSALLTPAEQTGTWKLKTQFNNNEYSQDLDAYNTKDKIGVELTGRTLADLTLLDSPIKVPLLLSEPINIIDALEMRDAVEKPQEFTIVAFVKYDKNQDVHSINLPFFETLQEYFERNRQTIIVVLENVQRNLKHINIDQFVRKYRAALGKLPQQANDYLNSFNWERQVSHAKEKLTALTKKYRITENDIQIALDDAKINFNEKLSQLQTYMIQFDQYIKDSYDLHDLKIAIANIIDEIIEKLKSLDEHYHIRVNLVKTIHDLHLFIENIDFNKSGSSTASWIQNVDTKYQIRIQIQEKLQQLKRHIQNIDIQHLAGKLKQHIEAIDVRVLLDQLGTTISFERINDVLEHVKHFVINPYWDFEVAEKINAFRAKVHELIERYEVDQHIQVLMDKLVELAHQYKLKETIQKLSNVLQQVKIKDYFEKLVGFID

SEQ ID NO: 8 - protein, 83 aa, Human
MRLFLSLPVLVVVLSIVLEGPAPAQGTPDVSSALDKLKEFGNTLEDKARELISRIKQSELSAKMREWFSETFQKVKEKLKIDS

SEQ ID NO: 9 - protein, 101 aa, Human
MGTRLLPALFLVLLVLGFEVQGTQQPQQDEMPSPTFLTQVKESLSSYWESAKTAAQNLYEKTYLPAVDEKLRDLYSKSTAAMSTYTGIFTDQVLSVLKGEE

SEQ ID NO: 10 - protein, 99 aa, Human
MQPRVLLVVALLALLASARASEAEDASLLSFMQGYMKHATKTAKDALSSVQESQVAQQARGWVTDGFSSLKDYWSTVKDKFSEFWDLDPEVRPASAVAA

SEQ ID NO: 11 - protein, 189 aa, Human
MVMLLLLLSALAGLFGAAEGQAFHLGKCPNPPVQENFDVNKYLGRWYEIEKIPTTFENGRCIQANYSLMENGKIKVLNQELRADGTVNQIEGEATPVNLTEPAKLEVKFSWFMPSAPYWILATDYENYALVYSCTCIIQLFHVDFAWILARNPNLPPETVDSLKNILTSNNIDVKKMTVTDQVNCPKLS

SEQ ID NO: 12 - protein, 98 aa, Human
GEATPVNLTEPAKLEVKFSWFMPSAPYWILATDYENYALVYSCTCIIQLFHVDFAWILARNPNLPPETVDSLKNILTSNNIDVKKMTVTDQVNCPKLS

SEQ ID NO: 13 - protein, 317 aa, Human [sequence omitted]

SEQ ID NO: 14 - protein, 345 aa, Human
MISPVLILFSSFLCHVAIAGRTCPKPDDLPFSTVVPLKTFYEPGEEITYSCKPGYVSRGGMRKFICPLTGLWPINTLKCTPRVCPFAGILENGAVRYTTFEYPNTISFSCNTGFYLNGADSAKCTEEGKWSPELPVCAPIICPPPSIPTFATLRVYKPSAGNNSLYRDTAVFECLPQHAMFGNDTITCTTHGNWTKLPECREVKCPFPSRPDNGFVNYPAKPTLYYKDKATFGCHDGYSLDGPEEIECTKLGNWSAMPSCKASCKVPVKKATVVYQGERVKIQEKFKNGMLHGDKVSFFCKNKEKKCSYTEDAQCIDGTIEVPKCFKEHSSLAFWKTDASDVKPC

SEQ ID NO: 15 - protein, 4548 aa, Human
MEHKEVVLLLLLFLKSAAPEQSHVVQDCYHGDGQSYRGTYSTTVTGRTCQAWSSMTPHQHNRTTENYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDAVAAPYCYTRDPGVRWEYCNLTQCSDAEGTAVAPPTVTPVPSLEAPSEQAPTEQRPGVQECYHGNGQSYRGTYSTTVTGRTCQAWSSMTPHSHSRTPEYYPNAGLIMNYCRNPDPVAAPYCYTRDPSVRWEYCNLTQCSDAEGTAVAPPTITPIPSLEAPSEQAPTEQRPGVQECYHGNGQSYQGTYFITVTGRTCQAWSSMTPHSHSRTPAYYPNAGLIKNYCRNPDPVAAPWCYTTDPSVRWEYCNLTRCSDAEWTAFVPPNVILAPSLEAFFEQALTEETPGVQDCYYHYGQSYRGTYSTTVTGRTCQAWSSMTPHQHSRTPENYPNAGLTRNYCRNPDAEIRPWCYTMDPSVRWEYCNLTQCLVTESSVLATLTVVPDPSTEASSEEAPTEQSPGVQDCYHGDGQSYRGSFSTTVTGRTCQSWSSMTPHWHQRTTEYYPNGGLTRNYCRNPDAEISPWCYTMDPNVRWEYCNLTQCPVTESSVLATSTAVSEQAPTEQSPTVQDCYHGDGQSYRGSFSTTVTGRTCQSWSSMTPHWHQRTTEYYPNGGLTRNYCRNPDAEIRPWCYTMDPSVRWEYCNLTQCPVMESTLLTTPTVVPVPSTELPSEEAPTENSTGVQDCYRGDGQSYRGTLSTTITGRTCQSWSSMTPHWHRRIPLYYPNAGLTRNYCRNPDAEIRPWCYTMDPSVRWEYCNLTRCPVTESSVLTTPTVAPVPSTEAPSEQAPPEKSPVVQDCYHGDGRSYRGISSTTVTGRTCQSWSSMIPHWHQRTPENYPNAGLTENYCRNPDSGKQPWCYTTDPCVRWEYCNLTQCSETESGVLETPTVVPVPSMEAHSEAAPTEQTPVVRQCYHGNGQSYRGTFSTTVTGRTCQSWSSMTPHRHQRTPENYPNDGLTMNYCRNPDADTGPWCFTMDPSIRWEYCNLTRCSDTEGTVVAPPTVIQVPSLGPPSEQDCMFGNGKGYRGKKATTVTGTPCQEWAAQEPHRHSTFIPGTNKWAGLEKNYCRNPDGDINGPWCYTMNPRKLFDYCDIPLCASSSFDCGKPQVEPKKCPGSIVGGCVAHPHSWPWQVSLRTRFGKHFCGGTLISPEWVLTAAHCLKKSSRPSSYKVILGAHQEVNLESHVQEIEVSRLFLEPTQADIALLKLSRPAVITDKVMPACLPSPDYMVTARTECYITGWGETQGTFGTGLLKEAQLLVIENEVCNHYKYICAEHLARGTDSCQGDSGGPLVCFEKDKYILQGVTSWGLGCARPNKPGVYARVSRFVTWIEGMMRNN

SEQ ID NO: 16 - protein, 3305 aa, Insect
MGKSNRLLSVLFVISVLWKAAYGNGKCQIACKGSSSPSFAAGQKYNYGVEGTVSVYLTGADNQETSLKMLGQASVSAISNCELELSVHNMVLSGPDGKKYPCPQGIEKPVRFSYQDGRVGPEICAAEDDSRRSLNIKRAIISLLQAEQKPSVQVDVFGVCPTEVSSSQEGGAVLLHRSRDLSRCAHREQGRNDFVNSIANPDAGIKDLQVLQSMLNVESKVNNGVPEKVSAIEEYLYKPFSVGENGARAKVHTKLTLSGKGGAGGGNAHCTESRSIIFDVPHGTSSASGNLNSVISAVKETARTVANDASSKSAGQFAQLVRIMRTSSKDDLMRIYSQVKAHQLEKRVYLDALLRAGTGESIEASIQILKSKDLSQLEQHLVFLSLGNARHVNNPALKAAAGLLDMPNLPKEVYLGAGALGGAYCREHDCHNVKPEGIVALSNKLGSKLQNCRPKNKPDEDVVVAILKGIRNIRHLEDSLIDKLVHCAVDNNVKARVRAVALEAFHADPCSAKIHKTAMDIMKNRQLDSEIRIKAYLAVIECPCSHSASEIKNLLDSEPVHQVGNFITSSLRHIRSSSNPDKQLAKKHYGQIRTPNKFKVDERKYSFYREMSYKLDALGAGGSVDQTVIYSQTSFLPRSVNFNLTVDLFGQSYNVMELGGRQGNLDRVVEHFLGPKSFLRTEDPQALYDNLVKRFQESKKKVEDSLSRGRRSIKSEIDVFDKNLKAESAPYNNELDLDIYVKLFGTDAVFLSFGDDKGFDFNKMLDQILGGCNSGINKAKHFQQEIRSHLLFMDAELAYPTSVGLPLRLNLIGAATARLDVATNIDIRQIFQSPQNAKADIKFVPSTDFEISGAFIIDADAFSTGIKVITNLHSSTGVHVNAKVLENGRGIDLQIGLPVDKQELIAASSDLVFVTAEKGQKEKQKVIKMEKGENEYSACFDQLSGPLGLTMCYDMVLPFPIVNRNDKLDSIAKAMGKWPLSGSAKFKLFLEKNDLRGYHIKAVVKEDKDAGRRSFELLLDTEGAKTRRSQLTGEAVYNENEVGVKLGLEAVGKVIYGHIWAHKKPNELVASVKGKLDDIEYSGKLGFSVQGNEHRAVYKPIFEYSLPDGSSPGSKKYEVKIDGQVIRECDGRVTKYTFDGVHVNLQNAEKPLEICGSVSTVAQPREVEFDVEVKHYASLKGSWKGSDVVLAFNNQLNPKINFDLKGKFENTDSMHNELDIHYGPNRGDNNARITFSQILKYHVENSKNFNVITKNNLEIRAVPFKLVANADVDPKKIDIDIEGQLQDKSAGFNLDARTHIKKEGDYSIKVKANLNNANLEAFSRRDIVNAEKSNVENYIDMKGVGRYELSGFVLHKTKPNDVNVGFIGHLKINGGGKNEDFKINIGHIETPAVFSSHATISGSRGDIIDYLLKIMRTANPNGNFKLVIKDSIAANGQYKVTDADGKGNGLIIIDFKKINRKIKGDVRFTAKEPVFNADIDLFLNFEKDNSDKVHFSTYNKKTDKVMDTKNKLEYAGKRTEVNIHQDGILAVTGKAHTVAELVLPTERCLSLKIDHDGAFKDGLYNGHMDMTISDAPKRGSGASTISYKGKVSNSNLDQEIIDYEGQINFKLKDGKNLQSTFSLKNNPDGDKFKYEFKSDVNGNLIPKPANLVATGTYSNSENEIDETYRLKGSYGSDIGFELAGVGTIKFLDAGDKKYLDDYTLTVRLPFEKAHDIKWVSTVLFLQPQGQEMTEYTLVESVQINADVYKIDANGKVGPKNGYGAVKVLVPHVEPFVLDYNYKSSHEGEKNNNYVELKTKYGKGKSASMVVDSSYAPHYSTLKVKANTPNNDKFKKLDVTVHSKNPSPDAYSNSVVVDADGRVYKIDSSIVLSKAHPVLDIQYHSPSSDKIRRLYLQGSSLSSTQGKLEVKVDNINDICLDAVSEANVQKDNVAFKVVANAKELGWKNYGIDISSKDSGSGKRLEFHATNDNKNVLSGSTSFISKQEGQKTIIEGSGSVKVKEEQKSANFKYIRTVFTDSNEKGVETFFNVALGERSYVAESRVTNYEYKNSYVYCEEKKQCAHAEIQSKIDMSTPGMIVNVINAGLDLRKLGVAPELGLQMRDEVSDRRPPRFTLDLHINKEDRKYHLHAYNTPENGHYASGVTVRLPSRVMALEYTLTHPTSQDLPFPIKGEACLDLDKNRPGHKTSARFLVDYSNSGSEDKAVAEIGFFHPKIEKEAVIRLNAFMKRPENGCFKIESSASLCHSALGTDRVAKVMFETTPNSVKFLADTPFVKAIDVEGSFNVNQQQRTQQCLFRICLLEGKPVQMSALVKDYQYYEFTTEESNRKLSYVGHLIPEKRVDISTDIILSGDKKNIAHGALFLQDNLVKSDYGLSKENFNYFLNALKKDLDTLEDRIKNVGEKASKDVEAVTQRAAPYFKKVEDNFRAEWNRFYQEIADDKVFKEISHVFNEIVQYIAKFIDEILQGTKRSWTPSCRPTLSHPRNREMYKKQIEPQVKQLYDTLGALMKEYLDGVIDVVAHFAAIVTDFFEKHKAELQELTNVFTEIFKDLTRLVVAQLKELPPKIAQIYNDIVSQITNMPFVVVLQEKWKEFNFAERAVQLVSQAYEAFSKILPTDELKEFAKALNAYLLKKIKEEKMEESKELPRAVREAGQRVLLITSIPALAVRRPRLRRWTWHHLKLAVGAGASAPSLGAASWSALRQLAAGDGPPALAPRGLPTAQLDPLDEVPNKLRAVVVNGQHIFTFDGRHLTFPGTCRYVLIHDHVDRNFTVLMQLANGQPKALVLEDKSGTIIELKDNGQVILNCQSHGFPVVEQDVFAFRQTSGRIGLCSKYGLMAFCTSKFEVCYFEVNGFYLGKLPGLLGDGNNEPYDDFRMPNGKICSSESEFGNSYRLSRSCPAANAPAHDHHQMHAPLPKPCERVFSGTSPLRPLSLMLDIAPFRQACIHAVTGADADKDLQQACDLARGYRRSRSRGCCPPRCPTPACAARTATGPGSWATPTSTNCPTDSLISSSPLRPLRTTPAHYKNMVVPLVSQLVDMLKGKHCTDIKVFLVGHTSKHPYPILYDTDLKLKNAKVSFDDKSRYDRIPFVKTGHEKFDSYSKTVVDFLNYIKIELGITNIEASQGQIFDLPLRPGAVKHVIFVTGGPTISQFFLLETVRALRNKVIIDEMAMSASLVTSTPGLKIGGGKNAAQIVGYEKHGVLLLGEKKQSKDSEAVRATLEVEDDPFSDAVEFANGVVFSASNYAALPAGQQKQFIQTAAHNIIQRMWREQIVQQCTCVFVDPFRVRSVCFNKARTEVARRRK

SEQ ID NO: 17 - protein, 386 aa, Insect
QQTFKNGVLESVKLGEEYKYVPFAKLNSGAQAKVTTKLTYTGTKAGAAPALTAGAPRSVIFENPQTDSQGNLETIKQELKTVVDSYSQNNVGKLTASHFTELVHLMRFSKKDDLLSLYQQVKAGNAHKNKLLARKVYFDALFRAGTGASVEALANLYKNKEVSDAKEQKLLFVSLNLVTSMTKPALKAAKLLLDGNPSREAYLSVGSLVNKYCQKFGCESADVKEISDKFSAKLGKCQPTTRQEEDTIVAVLKGIKNSNTLVAQLLDKVVGCASDKSSARVRVAAFQAYPAASCNKKIVNSALNFLKNVNEDSEIRIQAYLSPVECPSAAVANEIKALLDNEKVYQVGSFLTTHLASLRASADPTRDAARQHFANIRTTNQFPFDF

SEQ ID NO: 18 - protein, 189 aa, Insect
MAAKFVVVLAACVALSHSAMVRRDAPAGGNAFEEMEKHAKEFQKTFSEQFNSLVNSKNTQDFNKALKDGSDSVLQQLSAFSSSLQGAISDANGKAKEALEQARQNVEKTAEELRKAHPDVEKEANAFKDKLQAAVQTTVQESQKLAKEVASNMEETNKKLAPKIKQAYDDFVKHAEEVQKKLHEAATKQ

SEQ ID NO: 19 - protein, 212 aa, Synthetic
MGHHHHHHIEGRLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 20 - protein, 201 aa, Synthetic
MAKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 21 - protein, 414 aa, Synthetic
MGHHHHHHIEGRLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGTLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 22 - protein, 422 aa, Synthetic
MGHHHHHHIEGRLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGTGGGSGGGTLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 23 - protein, 168 aa, Synthetic
MGHHHHHHIEGRLKLLDNWDSVTSTFSKLREQLGPVTQEFWDNLEKETEGLRQEMSKDLEEVKAKVQPYLDDFQKKWQEEMELYRQKVEPYSDELRQRLAARLEALKENGGARLAEYHAKATEHLSTLSEKAKPALEDLRQGLLPVLESFKVSFLSALEEYTKKLNTQ

SEQ ID NO: 24 - protein, 168 aa, Synthetic
MGHHHHHHIEGRLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSARLAEYHAKATEHLSTLSEKAKPALEDLRQGLLPVLESFKVSFLSALEEYTKKLNTQ

SEQ ID NO: 25 - protein, 201 aa, Synthetic
MGHHHHHHHDYDIPTTENLYFQGSVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 26 - protein, 190 aa, Synthetic
MGHHHHHHHDYDIPTTENLYFQGKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 27 - protein, 179 aa, Synthetic
MGHHHHHHHDYDIPTTENLYFQGKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 28 - protein, 199 aa, Synthetic
MGHHHHHHHDYDIPTTENLYFQGSVTQEFWDNLEKETEGLRQEMSKDLEEVKAKVQPYLDDFQKKWQEEMELYRQKVEPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLL

SEQ ID NO: 29 - protein, 289 aa, Synthetic
MGHHHHHHHDYDIPTTENLYFQGLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 30 - protein, 278 aa, Synthetic
MGHHHHHHHDYDIPTTENLYFQGSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 31 - protein, 423 aa, Synthetic
MGHHHHHHHDYDIPTTENLYFQGLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEYTKKLNTQGTLKLLDNWDSVTSTFSKLREQLGPVTQEFWDNLEKETEGLRQEMKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 32 - protein, 401 aa, Synthetic
MGHHHHHHHDYDIPTTENLYFQGSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 33 - protein, 392 aa, Synthetic
MGHHHHHHHDYDIPTTENLYFQGSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGTPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 34 - protein, 397 aa, Synthetic
MGHHHHHHHDYDIPTTENLYFQGSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGTREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 35 - protein, 383 aa, Synthetic
MGHHHHHHHDYDIPTTENLYFQGSVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQNPGTPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 36 - protein, 379 aa, Synthetic
MGHHHHHHHDYDIPTTENLYFQGSVTQEFWDNLEKETEGLRQEMSKDLEEVKAKVQPYLDDFQKKWQEEMELYRQKVEPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLNPGTKDLEEVKAKVQPYLDDFQKKWQEEMELYRQKVEPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVTQEFWDNLEKETEGLRQEMS

SEQ ID NO: 37 - protein, 381 aa, Synthetic
MGHHHHHHHDYDIPTTENLYFQGSVTQEFWDNLEKETEGLRQEMSKDLEEVKAKVQPYLDDFQKKWQEEMELYRQKVEPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLSNPGTQKDLEEVKAKVQPYLDDFQKKWQEEMELYRQKVEPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVTQEFWDNLEKETEGLRQEMS

SEQ ID NO: 38 - protein, 1094 aa, Synthetic
MGHHHHHHIEGRLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGTLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQSTMGDSHEDTSATMPEAVAEEVSLFSTTDMVLFSLIVGVLTYWFIFRKKKEEIPEFSKIQTTAPPVKESSFVEKMKKTGRNIIVFYGSQTGTAEEFANRLSKDAHRYGMRGMSADPEEYDLADLSSLPEIDKSLVVFCMATYGEGDPTDNAQDFYDWLQETDVDLTGVKFAVFGLGNKTYEHFNAMGKYVDQRLEQLGAQRIFELGLGDDDGNLEEDFITWREQFWPAVCEFFGVEATGEESSIRQYELVVHEDMDVAKVYTGEMGRLKSYENQKPPFDAKNPFLAAVTANRKLNQGTERHLMHLELDISDSKIRYESGDHVAVYPANDSALVNQIGEILGADLDVIMSLNNLDEESNKKHPFPCPTTYRTALTYYLDITNPPRTNVLYELAQYASEPSEQEHLHKMASSSGEGKELYLSWVVEARRHILAILQDYPSLRPPIDHLCELLPRLQARYYSIASSSKVHPNSVHICAVAVEYEAKSGRVNKGVATSWLRAKEPAGENGGRALVPMFVRKSQFRLPFKSTTPVIMVGPGTGIAPFMGFIQERAWLREQGKEVGETLLYYGCRRSDEDYLYREELARFHKDGALTQLNVAFSREQAHKVYVQHLLKRDREHLWKLIHEGGAHIYVCGDARNMAKDVQNTFYDIVAEFGPMEHTQAVDYVKKLMTKGRYSLDVWS

SEQ ID NO: 39 - protein, 214 aa, Synthetic
MGHHHHHHHDYDIPTTENLYFQGSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGT

SEQ ID NO: 40 - protein, 212 aa, Synthetic
MGHHHHHHIEGCLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 41 - protein, 212 aa, Sythetic
MGHHHHHHIEGRLKLLDNWDSVTSTFSKLREQLGPVTQEFWDNLEKETEGLRQEMSKDLEEVKAKVQPYLDDFQKKWQEEMELYRQKVEPLRAELQEGARQCLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 42 - protein, 212 aa, Sythetic
MGHHHHHHIEGRLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHACATEHLSTLSEKAKPALEDLRQGLLPVLESFKVSFLSALEEYTKKLNTQ

(19) United States
(12) Patent Application Publication
(10) Pub. No.: US 2011/0195450 A1
(43) Pub. Date: Aug. 11, 2011

(54) In Vitro Protein Synthesis Systems for Membrane Proteins that Include Adolipoproteins and Phospholipid Adolipoprotein Particles

(75) Inventors: WIESLAW KUDLICKI, CARLSBAD, CA (US); JULIA FLETCHER, CARLSBAD, CA (US); FEDERICO KATZEN, San Marcos, CA (US)

(73) Assignee: LIFE TECHNOLOGIES CORPORATION, CARLSBAD, CA (US)

(21) Appl. No.: 12/848,017

(22) Filed: Jul. 30, 2010

Related U.S. Application Data
(63) Continuation of application No. 11/535,960, filed on Sep. 27, 2006, now abandoned.
(60) Provisional application No. 60/721,339, filed on Sep. 27, 2005.
(60) Provisional application No. 60/724,213, filed on Oct. 5, 2005.
(60) Provisional application No. 60/815,750, filed on Jun. 21, 2006.
(60) Provisional application No. 60/815,695, filed on Jun. 21, 2006.

(51) Int. Cl. C12P 21/00; C12N 9/00

(52) U.S. Cl. 435/68.1; 435/183

(57) ABSTRACT

In vitro protein synthesis systems and methods are provided that produce membrane proteins in soluble form. In some aspects, the invention provides methods of synthesizing proteins using in vitro protein synthesis systems that include an apolipoprotein, in which higher yields of soluble protein are produced than in the absence of the apolipoprotein. Apolipoproteins useful in the present invention include naturally occurring apolipoproteins, as well as sequence variants of wild-type apolipoproteins, and engineered apolipoproteins. The apolipoproteins can be provided in an in vitro protein synthesis system associated with lipid or not associated with lipid. The invention also provides compositions and kits for synthesis of proteins in soluble form, in which the compositions and kits include cell extracts for protein translation and at least one apolipoprotein biomolecule.

This application claims benefit of priority to U.S. Provisional Application 60/721,339, entitled “In vitro Translation Systems for Membrane Proteins that Include Phospholipid-Protein Particles”, filed Sep. 27, 2005; U.S. Provisional Application 60/724,213, entitled “In vitro Translation Systems for Membrane Proteins that Include Phospholipid-Protein Particles”, filed Oct. 4, 2005; U.S. Provisional Application 60/815,750, entitled “Cell-Free Protein Synthesis Systems Including Apolipoproteins”, filed Jun. 21, 2006; and U.S. Provisional Application 60/815,695, entitled “Cell-Free Protein Synthesis of Membrane Proteins Using Apolipoproteins”, filed Jun. 21, 2006; all of which are herein incorporated by reference in their entireties.

### SEQUENCE LISTING

The instant application contains a Sequence Listing which is hereby incorporated by reference in its entirety.

### BACKGROUND OF THE INVENTION

1. Field of the Invention

The invention relates generally to in vitro protein synthesis systems and more specifically to in vitro translation of membrane proteins.

2. Background Information

Strategies for treating medical conditions such as aging-related disorders, autoimmune diseases, and cancer rely heavily on understanding protein function. The majority of drug targets are proteins, and it is thought that at least half of protein drug targets are membrane proteins. The ability to efficiently synthesize proteins, and particularly membrane proteins, in amounts that can be used for studies of structure and function is critical to the discovery of new drugs that can combat disease.

In vitro protein synthesis systems, in which proteins can be made from a nucleic acid template in a cell free extract, allowing for efficient synthesis and subsequent isolation of proteins, can allow for high throughput structural and functional analysis of proteins that can accelerate research and drug discovery efforts in particular.

Unfortunately, not all proteins are synthesized in soluble form in in vitro synthesis systems. Membrane proteins in particular are often insoluble when produced in cell-free translation system, making it necessary to solubilize the proteins, often in denaturing detergents and then attempt to renature the proteins to investigate their native structure and activity. These endeavors are laborious and often unsuccessful.

Bayburt et al. have described the spontaneous formation of nanoscale lipid-protein particles when detergent solubilized apoliprotein A1 (“Apo A1”) and phospholipids are mixed (Bayburt, T. H., Carlson, J. W., and Sligar, S. G. (1998) “Reconstitution and Imaging of a Membrane Protein in a Nanometer-Sized Phospholipid Bilayer.” Journal of Structural Biology, 123, 37-44.) Dialyzing away the detergent leaves nanoscale lipid-protein particles that, by structural analysis have been determined to be composed of a lipid bilayer encircled by the Apo A1 protein. Bayburt and Sligar have described synthetic variants of Apo A1 (“scaffold proteins”) that behave like Apo A1 in forming lipid-protein particles in the presence of detergent. (Civjan, N. R., Bayburt, T. H., Schuler, M. A., and Sligar, S. G. (2003) “Direct Solubilization of Heterologously Expressed Membrane Proteins by Incorporation into Nanoscale Lipid Bilayers.” BioTechniques, 35, 556-563 U.S. Pat. No. 7,048,949, and U.S. Patent Application Publication No. 2005/0182243, all of which are herein incorporated by reference in their entireties. These researchers have found that other membrane proteins, when solubilized with detergent, will incorporate into the lipid bilayer of the nanodiscs if provided in the same self-assembly detergent mix and then subjected to dialysis.

This technology for providing a membrane protein in soluble form however still requires a large effort in purifying and solubilizing the membrane protein before it is combined with the nanodisc components in the self-assembly detergent mix. These processes must be individualized for particular proteins, are time-consuming and labor-intensive, and often require the use of harsh denaturing reagents that can affect protein function. Thus, a need exists for a convenient method of expressing membrane proteins in in vitro systems that provide the protein in a soluble, native, and substantially purified or readily purifiable form using faster procedures.

### SUMMARY OF THE INVENTION

The present invention provides efficient systems and methods for synthesizing proteins in cell-free in vitro synthesis systems that include apolipoproteins, including engineered apolipoproteins and variants of naturally-occurring apolipoproteins. In its various aspects and embodiments, the present invention provides efficient systems and methods for synthesizing membrane proteins in a cell-free system in soluble form.

In one aspect, the invention provides a method of synthesizing a protein of interest in vitro, in which the method includes: adding a nucleic acid template that encodes a protein of interest to an in vitro protein synthesis system that includes an apolipoprotein, and incubating the in vitro protein synthesis system to synthesize the protein of interest. In some preferred embodiments, the protein of interest is synthesized in soluble form. In some preferred embodiments, a protein of interest translated using the methods of the invention is a membrane protein.

An apolipoprotein used in the methods of the invention can be any apolipoprotein, such as but not limited to: Apolipoprotein A-I, Apolipoprotein A-II, Apolipoprotein A-IV, Apolipoprotein A-V, Apolipoprotein B-100, Apolipoprotein B-48, Apolipoprotein C-I, Apolipoprotein C-II, Apolipoprotein C-III, Apolipoprotein D, Apolipoprotein E, Apolipoprotein H, Lipoprotein (a), Apoliphorin I, Apoliphorin II, or Apoliphorin III, a variant of any of the aforementioned apolipoproteins, or an apolipoprotein engineered using one or more domain sequences of a naturally occurring apolipoprotein, or sequences substantially homologous thereto.

The invention includes, in some embodiments, the use of apolipoprotein variants or engineered apolipoproteins with 70% or greater amino acid sequence identity with at least 15 consecutive amino acids of an apolipoprotein such as but not limited to Apolipoprotein A-I, Apolipoprotein A-II, Apolipoprotein A-IV, Apolipoprotein A-V, Apolipoprotein B-100, Apolipoprotein B-48, Apolipoprotein C-I, Apolipoprotein C-II, Apolipoprotein C-III, Apolipoprotein D, Apolipoprotein E, Apolipoprotein H, Lipoprotein (a), Apoliphorin I, Apoliphorin II, or Apoliphorin III.

The invention includes, in some embodiments, the use of apolipoprotein variants or engineered apolipoproteins with 90% or greater amino acid sequence identity with at least 10 consecutive amino acids of a helical domain of an apolipoprotein such as but not limited to Apolipoprotein A-I, Apolipoprotein A-II, Apolipoprotein A-IV, Apolipoprotein A-V, Apolipoprotein B-100, Apolipoprotein B-48, Apolipoprotein C-I, Apolipoprotein C-II, Apolipoprotein C-III, Apolipoprotein D, Apolipoprotein E, Apolipoprotein H, Lipoprotein (a), Apoliphorin I, Apoliphorin II, or Apoliphorin III.

An apolipoprotein added to an in vitro synthesis system can have an amino acid sequence that is modified with respect to the amino acid sequence of a wild-type apolipoprotein by having one or more amino acid deletions, insertions, or substitutions. An apolipoprotein added to an in vitro synthesis system can have one or more chemical or enzymatic modifications. In some embodiments, an apolipoprotein added to an in vitro synthesis system comprises a label or tag, such as a peptide tag.

In some preferred embodiments, a protein of interest translated using the methods of the invention is a membrane protein, and after incubating the in vitro protein synthesis system a larger amount of the protein of interest is synthesized in soluble form than when the protein is translated in the absence of the apolipoprotein. In some preferred embodiments, a protein of interest translated using the methods of the invention is a membrane protein, and after incubating the in vitro protein synthesis system there is a higher percentage of soluble protein of interest to total protein of interest synthesized than when the protein of interest is translated in the absence of the apolipoprotein.

In some embodiments of the methods of the invention, following synthesis of a protein of interest in the presence of an apolipoprotein, the protein of interest is associated with the apolipoprotein. In some embodiments, a protein of interest synthesized in vitro in the presence of an apolipoprotein co-isolates with the apolipoprotein.

In some embodiments of the methods of the invention, an apolipoprotein provided in an in vitro protein synthesis system is a present in a phospholipid-apolipoprotein particle. In some embodiments of the methods of the invention, an apolipoprotein in vitro protein synthesis system is present in a phospholipid-apolipoprotein particle and a protein of interest synthesized in the system becomes associated with the phospholipid-apolipoprotein particle. In some preferred embodiments, a protein of interest synthesized in an in vitro reaction that includes a phospholipid-apolipoprotein particle can be isolated with the phospholipid-apolipoprotein particle.

In some embodiments of the invention, the methods further include isolating the protein of interest from the in vitro synthesis mixture. Isolation can be, for example, by means of a peptide tag that is part of the protein of interest, or by a peptide tag that is part of the apolipoprotein provided in the in vitro protein synthesis reaction.

In another aspect, the invention provides an in vitro protein synthesis system that includes a cell extract and an apolipoprotein. Cell extracts that include components of the protein synthesis machinery are well-known in the art, and can be from prokaryotic or eukaryotic cells. An apolipoprotein used in the methods of the invention can be any apolipoprotein, including but not limited to: Apolipoprotein A-I, Apolipoprotein A-II, Apolipoprotein A-IV, Apolipoprotein A-V, Apolipoprotein B-100, Apolipoprotein B-48, Apolipoprotein C-I, Apolipoprotein Apolipoprotein Apolipoprotein D, Apolipoprotein E, Apolipoprotein H, Lipoprotein (a), Apoliphorin I, Apoliphorin II, or Apoliphorin III, a variant of any of these apolipoproteins, or an engineered apolipoprotein having at least one domain with substantial homology to a naturally-occurring apolipoprotein, as described herein.

An apolipoprotein provided in an in vitro synthesis system can be a modified or derivatized apolipoprotein, in which the modified or derivatized apolipoprotein has one or more chemical modifications. An apolipoprotein provided in an in vitro synthesis system can comprise a tag or label.

The in vitro protein synthesis system preferably includes at least one chemical energy source for providing the energy for protein synthesis. Nonlimiting examples of energy sources are nucleotides, such as ATP or GTP, glycolytic intermediates, phosphorylated compounds, and energy-generating enzymes. In vitro protein synthesis systems of the invention can further comprise free amino acids, salts, buffering compounds, enzymes, inhibitors, or cofactors.

The in vitro protein synthesis system can further include one or more nucleic acid templates. A nucleic acid template can be a DNA template or an RNA template, and can encode any protein of interest whose in vitro synthesis is desired. A nucleic acid template present in an in vitro protein synthesis system can encode more than one protein of interest. A nucleic acid template in an IVPS system can be bound to a solid support, such as, for example, a bead, matrix, chip, array, membrane, sheet, dish, or plate.

In vitro protein synthesis systems of the invention can further comprise one or more detergents or one or more lipids, such as but not limited to one or more phospholipids. In some exemplary embodiments, an in vitro synthesis system of the invention can include an apolipoprotein associated with one or more lipids. In some exemplary embodiments, an in vitro synthesis system of the invention includes an apolipoprotein associated with one or more phospholipids in a phospholipid-apolipoprotein particle. In these embodiments, a protein of interest synthesized in the in vitro synthesis system preferably becomes associated with the phospholipid-apolipoprotein particle. In preferred embodiments, a protein of interest synthesized in the in vitro synthesis system can be isolated with the phospholipid-apolipoprotein particle.

In yet another aspect, the invention provides a method of synthesizing a protein in vitro, in which the method includes: adding to an in vitro synthesis system a nucleic acid construct that encodes an apolipoprotein and a nucleic acid construct that encodes a protein of interest, and incubating the in vitro protein synthesis system to synthesize an apolipoprotein and a protein of interest. In some preferred embodiments, the protein of interest is synthesized in soluble form. In some preferred embodiments, the protein of interest is a membrane protein.

In some embodiments, an apolipoprotein is provided on a first nucleic acid construct, and a protein of interest is provided on a second nucleic acid construct. In other embodiments of this aspect of the invention, sequences encoding an apolipoprotein and sequences encoding a protein of interest are provided on the same nucleic acid construct. A DNA construct that includes sequences encoding an apolipoprotein and sequence encoding a protein of interest can include separate promoters for the two gene sequences, and/or can include an IRES sequence between the two gene sequences.

In these aspects of the present invention, a nucleic acid construct encoding an apolipoprotein can encode any apolipoprotein, such as but not limited to: Apolipoprotein A-I, Apolipoprotein A-II, Apolipoprotein A-IV, Apolipoprotein A-V, Apolipoprotein B-100, Apolipoprotein B-48, Apolipoprotein C-I, Apolipoprotein C-II, Apolipoprotein C-III, Apolipoprotein D, Apolipoprotein E, Apolipoprotein H, Lipoprotein (a), Apoliphorin I, Apoliphorin II, or Apoliphorin III, or a variant of Apolipoprotein A-I, Apolipoprotein A-II, Apolipoprotein A-IV, Apolipoprotein A-V, Apolipoprotein B-100, Apolipoprotein B-48, Apolipoprotein C-I, Apolipoprotein C-II, Apolipoprotein C-III, Apolipoprotein D, Apolipoprotein E, Apolipoprotein H, Lipoprotein (a), Apoliphorin I, Apoliphorin II, or Apoliphorin III, a variant of any of these apolipoproteins, or an engineered apolipoprotein having at least one domain with substantial homology to a naturally-occurring apolipoprotein, as described herein.

A nucleic acid construct encoding an apolipoprotein can encode an apolipoprotein having an amino acid sequence that is modified with respect to the amino acid sequence of a wild-type apolipoprotein. In some embodiments, a nucleic acid construct encoding an apolipoprotein or apolipoprotein variant encodes a tag sequence fused to the apolipoprotein sequence.

In some preferred embodiments, a protein of interest translated using the methods of the invention is a membrane protein, and after incubating the in vitro protein synthesis system, a larger amount of the protein of interest is synthesized in soluble form than when the protein is translated in the absence of apolipoprotein translation in the same reaction. In some preferred embodiments, a protein of interest translated using the methods of the invention is a membrane protein, and after incubating the in vitro protein synthesis system there is a higher percentage of soluble protein of interest to total protein of interest is synthesized than when the protein of interest is translated in the absence of the apolipoprotein being translated in the same reaction.

In some embodiments, an in vitro protein synthesis system of the invention that comprises nucleic acid construct(s) encoding a protein of interest and an apolipoprotein comprises one or more lipids, such as but not limited to one or more phospholipids. In some embodiments, methods of the invention that comprise synthesizing a protein of interest in soluble form comprise adding to an in vitro synthesis system that comprises at least one lipid a nucleic acid construct that encodes an apolipoprotein and a nucleic acid construct that encodes a protein of interest and incubating the in vitro protein synthesis system to synthesize an apolipoprotein particle and a protein of interest associated with the phospholipid-apolipoprotein particle. In these methods the nucleic acid sequences encoding the apolipoprotein can be included on the same nucleic acid molecule as the sequences encoding the protein of interest, or the apolipoprotein and protein of interest synthesized in the in vitro protein synthesis reaction can be encoded on separate nucleic acid molecules.

In some embodiments of these aspects of the invention, the methods further include isolating the protein of interest from the in vitro synthesis mixture. Isolation can be performed, for example, by using an affinity reagent that binds a tag incorporated into the sequence of the apolipoprotein or the protein of interest.

The invention also provides, in a further aspect, an in vitro protein synthesis system that includes a cell extract, a nucleic acid template that encodes an apolipoprotein, and a nucleic acid template that encodes a protein of interest. In certain embodiments, the invention includes an in vitro protein synthesis system that includes a cell extract, a first nucleic acid molecule that encodes an apolipoprotein, and a second nucleic acid molecule that encodes a protein of interest. In other embodiments, an in vitro protein synthesis system that includes a cell extract and a nucleic acid template that encodes an apolipoprotein and a protein of interest.

An apolipoprotein encoded by a nucleic acid template used in the in vitro systems of the invention can be any apolipoprotein, such as but not limited to: An apolipoprotein used in the methods of the invention can be any apolipoprotein, such as but not limited to: Apolipoprotein A-I, Apolipoprotein A-II, Apolipoprotein A-IV, Apolipoprotein A-V, Apolipoprotein B-100, Apolipoprotein B-48, Apolipoprotein C-I, Apolipoprotein C-II, Apolipoprotein C-III, Apolipoprotein D, Apolipoprotein E, Apolipoprotein H, Lipoprotein (a), Apoliphorin I, Apoliphorin II, or Apoliphorin III, a variant of any of these apolipoproteins, or an engineered apolipoprotein having at least one domain with substantial homology to a naturally-occurring apolipoprotein, as described herein.

An apolipoprotein sequence encoded by a nucleic acid construct used in the methods and in vitro synthesis systems of the invention can be modified with respect to the sequence of a naturally-occurring or wild-type sequence, and can have one or more deletions, mutations, or additional sequences with respect to a wild-type apolipoprotein sequence. A construct that encodes an apolipoprotein can also encode an amino acid tag fused in frame with the apolipoprotein sequence. A nucleic acid template that encodes an apolipoprotein can be a DNA template or an RNA template. A nucleic acid template that encodes an apolipoprotein can be bound to a solid support, such as, for example, a bead, matrix, chip, array, membrane, sheet, dish, or plate.

A nucleic acid template that encodes a protein of interest can be a DNA template or an RNA template, and can encode any protein of interest, such as but not limited to: an enzyme, structural protein, carrier protein, hormone, growth factor, inhibitor, or activator. In some preferred embodiments, a protein of interest translated using the methods of the invention is a membrane protein. A construct that encodes a protein of interest can also encode an amino acid tag fused in frame with the protein of interest sequence.

A nucleic acid construct present in an in vitro protein synthesis system of the invention can encode more than one protein of interest. A nucleic acid template that encodes a protein of interest can be bound to a solid support, such as, for example, a bead, matrix, chip, array, membrane, sheet, dish, or plate.

A single nucleic acid construct present in an in vitro synthesis system of the invention can encode both an apolipoprotein and a protein of interest. In these embodiments, the invention provides an in vitro protein synthesis system that comprises a cell extract, an energy source, a nucleic acid template that encodes an apolipoprotein, and a nucleic acid template that encodes an apolipoprotein and a protein of interest.

In vitro protein synthesis systems of the invention can further comprise at least one chemical energy source, free amino acids, salts, enzymes, inhibitors, or cofactors. In vitro protein synthesis systems of the invention can further comprise one or more detergents or one or more lipids, such as but not limited to one or more phospholipids.

Kits are also provided in the invention, in which the kits include a cell extract and at least one apolipoprotein or at least one nucleic acid encoding an apolipoprotein. A kit can optionally further include one or more of: a solution of one or more amino acids, one or more buffers, one or more salts, one or more nucleotides, one or more enzymes, one or more inhibitors, one or more energy sources, one or more lipids, one or more detergents, one or more nucleic acid vectors, or one or more nucleic acid constructs.

In one embodiment of a kit of the invention, a kit is provided for in vitro protein synthesis that includes a cell extract and at least one apolipoprotein. The apoplipoprotein can be present in the cell extract, or can be provided separately as a solid or in solution. In another embodiment of a kit of the invention, a cell extract and at least one nucleic acid construct encoding an apolipoprotein are provided. The nucleic acid construct can be an RNA construct or a DNA construct and can be provided as a solid, such as a lypophilate, or in solution.

In another embodiment of a kit of the invention, a kit is provided for in vitro protein synthesis that includes a cell extract and at least one phospolipid-apolipoprotein particle composition. The phospholipid-apoplipoprotein particle composition can be present in the cell extract, or can be provided separately.

The invention described herein is not limited to specific compositions or process steps, as such may vary. Section headings provided herein are for convenience only, and are not intended to limit the scope of the invention.

### BRIEF DESCRIPTION OF THE DRAWINGS

FIG. 1a) depicts a histogram of GFP produced in IVPS reactions in the presence (right column of each pair) and absence (left column of each pair) of 4 micromolar and 40 micromolar PAPs. b) is an autorad showing total and soluble yield of GFP synthesized in IVPS reactions in the absence and presence of PAPs.

FIG. 2a) is a histogram showing total and soluble bacterial EmrE expression in the absence and presence of 20 micromolar PAPs. b) is a histogram showing total and soluble mammalian potassium channel protein expression in the absence and presence of 4 micromolar PAPs.

FIG. 3a) depicts Coomassie stained gels of PAPs in which the engineered apolipoprotein was his tagged, purified on Ni-NTA resin. Lanes 5-8 are eluted fractions. b) depicts Coomassie stained gels of a translation of EmrE protein (no PAPs), elution after binding on Ni-NTA resin. Lanes 5-8 are eluted fractions c) depicts Coomassie stained gels of EmrE protein translated with PAPs in which the engineered apolipoprotein was his tagged and purified on Ni-NTA resin. Lanes 5-8 are eluted fractions.

FIG. 4 is an autoradiogram of EmrE protein translated in the presence of PAPs (lanes 3 and 4) and absence of PAPs (lane 2) and GFP translated in the presence (lane 6) or absence (lane 5) of PAPs.

FIG. 5 provides autorads of gels showing purification on Ni-NTA of GFP translated in the a) presence and b) absence of PAPs having a his-tagged engineered apolipoprotein, and of MscL translated in the c) presence and d) absence of PAPs having a his-tagged engineered apolipoprotein.

FIG. 6 provides autorads of gels of translations of a) and b) GFP translated in the presence of PAPs and c) EmrE translated in the presence of PAPs.

FIG. 7a) shows lumio detection EmrE with a lumio sequence synthesized in translation reactions that contained PAPs. b) shows lumio detection of lumio-tagged EmrE made in translation reactions that did not include PAPs.

FIG. 8 depicts a histogram showing luciferase activity following translation of luciferase in reactions having increasing amounts of PAPs.

FIG. 9 is an autoradiogram of a native gel of rabbit reticulocyte translation products of reactions that contained or did not contain PAPs.

FIG. 10 is an autoradiogram of a native gel of rabbit reticulocyte translation products of reactions that contained or did not contain PAPs.

FIG. 11 provides autoradiographs of gels on which translation products of in vitro protein synthesis reactions that either contained or lacked Apo A-I were electrophoresed. (A) Yield of total bacterial EmrE protein is not affected by the presence of apolipoprotein or phospholipids (lanes 1-4), while soluble yield of EmrE protein is enhanced by the presence of apolipoprotein in the in vitro protein synthesis reaction (lanes 5-8). (B) Yield of total mammalian GABA A protein is not affected by the presence of apolipoprotein or phospholipids (lanes 1-4), while soluble yield of GABA A protein is enhanced by the presence of apolipoprotein in the in vitro protein synthesis reaction (lanes 5-8).

FIG. 12 provides autoradiographs of gels on which translation products of in vitro protein synthesis reactions that either lacked Apo A-I or contained different amounts of Apo A-I were electrophoresed. (A) Yield of total bacterial EmrE protein is not affected by the presence of apolipoprotein (lanes 1-4), while soluble yield of EmrE protein is enhanced by the presence of apolipoprotein in the in vitro protein synthesis reaction (lanes 5-8). (B) Yield of total mammalian GABA A protein is not affected by the presence of apolipoprotein (lanes 1-4), while soluble yield of GABA A protein is enhanced by the presence of apolipoprotein in the in vitro protein synthesis reaction (lanes 5-8).

FIG. 13 provides a stained gel and autoradiograph of the gel on which his-tagged and 35S-labeled EmrE translation products of in vitro protein synthesis reactions that included Apo A-I were electrophoresed after Ni-NTA column isolation. (A) The column fractions show that ApoA1 and EmrE co-elute, (B) the autoradiograph confirms the presence of EmrE in the eluted fractions.

### DETAILED DESCRIPTION

Definitions

Unless defined otherwise, all technical and scientific terms used herein have the same meaning as commonly understood by one of ordinary skill in the art to which this invention is related. The following terms are defined for purposes of the invention as described herein. The singular form “a”, “an” and “the” include plural referents unless the context clearly dictates otherwise. Thus, for example, reference to “a ligand” includes a plurality of ligands and reference to “an antibody” includes a plurality of antibodies, etc.

As used herein, the terms “about” or “approximately” when referring to any numerical value are intended to mean a value of ±10% of the stated value. For example, “about 50° C.” (or “approximately 50° C.”) encompasses a range of temperatures from 45° C. to 55° C., inclusive. Similarly, “about 100 mM” (or “approximately 100 mM”) encompasses a range of concentrations from 90 mM to 110 mM, inclusive.

The terms “in vitro protein synthesis” (IVPS), “in vitro translation”, “cell-free translation”, “RNA template-driven in vitro protein synthesis”, “RNA template-driven cell-free protein synthesis” and “cell-free protein synthesis” are used interchangeably herein and are intended to refer to any method for cell-free synthesis of a protein. In vitro transcription-translaction (NTT) is one non-limiting example of IVPS.

The terms “in vitro transcription” (IVT) and “cell-free transcription” are used interchangeably herein and are intended to refer to any method for cell-free synthesis of RNA from DNA without synthesis of protein from the RNA. A preferred RNA is messenger RNA (mRNA), which encodes proteins.

The terms “in vitro transcription-translation” (PITT), “cell-free transcription-translation”, “DNA template-driven in vitro protein synthesis” and “DNA template-driven cell-free protein synthesis” are used interchangeably herein and are intended to refer to any method for cell-free synthesis of mRNA from DNA (transcription) and of protein from mRNA (translation).

As used herein, the term “gene” refers to a nucleic acid that contains information necessary for expression of a polypeptide, protein, or untranslated RNA (e.g., rRNA, tRNA, anti-sense RNA). When the gene encodes a protein, it includes the promoter and the structural gene open reading frame sequence (ORF), as well as other sequences involved in expression of the protein. When the gene encodes an untranslated RNA, it includes the promoter and the nucleic acid that encodes the untranslated RNA.

As used herein, the phrase “nucleic acid molecule” refers to a sequence of contiguous nucleotides (riboNTPs, dNTPs, ddNTPs, or combinations thereof) of any length. A nucleic acid molecule may encode a full-length polypeptide or a fragment of any length thereof, or may be non-coding. As used herein, the terms “nucleic acid molecule” and “polynucleotide” may be used interchangeably and include both RNA and DNA.

“Operably linked” refers to a juxtaposition wherein the components so described are in a relationship permitting them to function in their intended manner. For example, a control sequence operably linked to a coding sequence is positioned in such a way that expression of the coding sequence is achieved under conditions compatible with control sequences.

As used herein, the term “polypeptide” refers to a sequence of contiguous amino acids of any length. The terms “peptide,” “oligopeptide,” or “protein” may be used interchangeably herein with the term “polypeptide.”

A “mutation” is a change in the genome with respect to the standard wild-type sequence. Mutations can be deletions, insertions, or rearrangements of nucleic acid sequences at a position in the genome, or they can be single base changes at a position in the genome, referred to as “point mutations”.

A “substitution,” as used herein, refers to the replacement of one or more amino acids or nucleotides by different amino acids or nucleotides, respectively.

A “variant” of a polypeptide or protein, as used herein, refers to an amino acid sequence that is altered with respect to the referenced polypeptide or protein by one or more amino acids. Preferably a variant of a polypeptide retains at least one activity of the polypeptide. Preferably a variant of a polypeptide has at least 60% identity to the referenced protein over a sequence of at least 15 amino acids. More preferably a variant of a polypeptide is at least 70% identical to the referenced protein over a sequence of at least 15 amino acids. Protein variants can be, for example, at least 80%, at least 90%, at least 95%, or at least 99% identical to referenced polypeptide over a sequence of at least 15 amino acids. Protein variants of the invention can be, for example, at least 80%, at least 90%, at least 95%, or at least 99% identical to referenced polypeptide over a sequence of at least 20 amino acids. The variant may have “conservative” changes, wherein a substituted amino acid has similar structural or chemical properties (e.g., replacement of leucine with isoleucine). A variant may also have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological or immunological activity may be found using computer programs well known in the art, for example, DNASTAR software.

“Conservative amino acid substitutions” are those substitutions that are predicted to least interfere with the properties of the original protein, i.e., the structure and especially the function of the protein is conserved and not significantly changed by such substitutions. Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain. Conservative substitutions include: the exchange of one negatively charged amino acid for another, where negatively charged amino acids may include aspartic acid and glutamic acid; the exchange of one positively charged amino acid for another, where one positively charged amino acids include lysine and arginine; and the exchange of amino acids with uncharged polar head groups having similar hydrophilicity values, where one group of amino acids with similar hydrophobicity may include leucine, isoleucine, and valine, another group may include glycine and alanine, a third group may include asparagine and glutamine, a fourth group may include serine and threonine, and a fifth group may include phenylalanine and tyrosine.

A “deletion” refers to a change in the amino acid or nucleotide sequence that results in the absence of one or more amino acid residues or nucleotides.

The term “derivative” refers to a chemically modified polynucleotide or polypeptide. Chemical modifications of a polynucleotide can include, for example, replacement of hydrogen by an alkyl, acyl, hydroxyl, or amino group. A derivative polynucleotide encodes a polypeptide which retains at least one biological or immunological function of the natural molecule. A derivative polypeptide is one modified by glycosylation, pegylation, biotinylation, or any similar process that retains at least one biological or immunological function of the polypeptide from which it was derived.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

Percent identity between polypeptide sequences may be determined using the default parameters of the CLUSTAL V algorithm as incorporated into the MEGALIGN version 3.12e sequence alignment program (described and referenced above). For pairwise alignments of polypeptide sequences using CLUSTAL V, the default parameters are set as follows: Ktuple=1, gap penalty=3, window=5, and “diagonals saved”=5. The PAM250 matrix is selected as the default residue weight table. As with polynucleotide alignments, the percent identity is reported by CLUSTAL V as the “percent similarity” between aligned polypeptide sequence pairs.

Alternatively the NCBI BLAST software suite may be used. For example, for a pairwise comparison of two polypeptide sequences, one may use the “BLAST 2 Sequences” tool Version 2.0.12 (Apr. 21, 2000) or a later version, such as Version 2.2.12 released Aug. 28, 2005; 2.2.13 released Dec. 6, 2005, or 2.2.14, released May 7, 2006, with blastp set at default parameters. Such default parameters may be, for example: Matrix: BLOSUM62; Open Gap: 11 and Extension Gap: 1 penalties; Gap x drop-off. 50; Expect: 10; Word Size: 3; Filter: on.

“Substantially purified” refers to the state of a species or activity that is the predominant species or activity present (for example on a molar basis it is more abundant than any other individual species or activities in the composition) and preferably a substantially purified fraction is a composition wherein the object species or activity comprises at least about 50 percent (on a molar, weight or activity basis) of all macromolecules or activities present. Generally, a substantially pure composition will comprise more than about 80 percent of all macromolecular species or activities present in a composition, more preferably more than about 85%, 90%, or 95%.

The terms “detectably labeled” and “labeled” are used interchangeably herein and are intended to refer to situations in which a molecule (e.g., a nucleic acid molecule, protein, nucleotide, amino acid, and the like) have been tagged with another moiety or molecule that produces a signal capable of being detected by any number of detection means, such as by instrumentation, eye, photography, radiography, and the like. In such situations, molecules can be tagged (or “labeled”) with the molecule or moiety producing the signal (the “label” or “detectable label”) by any number of art-known methods, including covalent or ionic coupling, aggregation, affinity coupling (including, e.g., using primary and/or secondary antibodies, either or both of which may comprise a detectable label), and the like. Suitable detectable labels for use in preparing labeled or detectably labeled molecules in accordance with the invention include, for example, heavy isotope labels, heavy atom labels, radioactive isotope labels, fluorescent labels, chemiluminescent labels, bioluminescent labels and enzyme labels, and others that will be familiar to those of ordinary skill in the art.

The term “label” as used herein refers to a chemical moiety or protein that is directly or indirectly detectable (e.g. due to its spectral properties, conformation or activity) when attached to a target or compound and used in the present methods. The label can be directly detectable (fluorophore) or indirectly detectable (hapten or enzyme). Such labels include, but are not limited to, radiolabels that can be measured with radiation-counting devices; pigments, dyes or other chromogens that can be visually observed or measured with a spectrophotometer; spin labels that can be measured with a spin label analyzer; heavy atom labels used, for example, in X-ray crystallography and NMR; heavy isotope labels used, for example, in mass spectrometry; and fluorescent labels (fluorophores), where the output signal is generated by the excitation of a suitable molecular adduct and that can be visualized by excitation with light that is absorbed by the dye or can be measured with standard fluorometers or imaging systems, for example. The label can be a chemiluminescent substance, where the output signal is generated by chemical modification of the signal compound; a metal-containing substance; or an enzyme, where there occurs an enzyme-dependent secondary generation of signal, such as the formation of a colored product from a colorless substrate. In the context of the present invention, the term “label” specifically includes naturally occurring amino acids, such as amino acids that might be weakly fluorescent (e.g., tryptophan) or absorb in the UV. Such amino acids are not intended to be encompassed by the term “label” or “detectable label”. The term label can also refer to a “tag” or hapten that can bind selectively to a conjugated molecule such that the conjugated molecule, when added subsequently along with a substrate, is used to generate a detectable signal. For example, one can use biotin as a tag and then use an avidin or streptavidin conjugate of horseradish peroxidate (HRP) to bind to the tag, and then use a colorimetric substrate (e.g., tetramethylbenzidine (TMB)) or a fluorogenic substrate such as Amplex Red reagent (Molecular Probes, Inc.) to detect the presence of HRP. Numerous labels are know by those of skill in the art and include, but are not limited to, particles, fluorophores, haptens, enzymes and their colorimetric, fluorogenic and chemiluminescent substrates and other labels that are described in RICHARD P. HAUGLAND, MOLECULAR PROBES HANDBOOK OF FLUORESCENT PROBES AND RESEARCH PRODUCTS (9^{th} edition, CD-ROM, September 2002), supra.

A “tag” or an “amino acid sequence tag” is a series of amino acids that can be specifically bound by an affinity reagent. Examples of tags that can be incorporated into proteins for capture or detection of the protein using an affinity reagent include, without limitation, his tags comprising multiple (four or more, typically six) histidines, FLAG tag, Hemaglutinin tag, myc tag, glutathione-S-transferase, maltose binding protein, calmodulin, chitin binding protein, etc. Another amino acid sequence tag is a tetracysteine-containing lumio tag that can be used for purification or detection of a protein using a tetraaresenical or biarsenical reagent (see, e.g., U.S. Pat. Nos. 6,054,271; 6,008,378; 5,932,474; 6,451,569; WO 99/21013, which are incorporated into the present disclosure by reference).

A “solid support” is a solid material having a surface for attachment of molecules, compounds, cells, or other entities. A solid support can be a chip or array that comprises a surface, and that may comprise glass, silicon, nylon, polymers, plastics, ceramics, or metals. A solid support can also be a membrane, such as a nylon, nitrocellulose, or polymeric membrane, or a plate or dish and can be comprised of glass, ceramics, metals, or plastics, such as, for example, a 96-well plate made of, for example, polystyrene, polypropylene, polycarbonate, or polyallomer. A solid support can also be a bead or particle of any shape, and is preferably spherical or nearly spherical, and preferably a bead or particle has a diameter or maximum width of 1 millimeter or less, more preferably of between 0.1 to 100 microns. Such particles or beads can be comprised of any suitable material, such as glass or ceramics, and/or one or more polymers, such as, for example, nylon, polytetrafluoroethylene, TEFLON.™., polystyrene, polyacrylamide, sepaharose, agarose, cellulose, cellulose derivatives, or dextran, and/or can comprise metals, particularly paramagnetic metals, such as iron.

As used herein “associated with” means directly or indirectly bound to. A first biomolecule that is associated with s second biomolecule can be co-isolated with the second biomolecule using at least one capture or separation procedure that is based on the binding or mobility properties of the second biomolecule.

A “phosphophospholipid-apolipoprotein particle” is a molecular complex that includes at least one apolipoprotein and at least one phospholipid, in which the phospholipid is arranged in a bilayer, and typically in a discoidal shape of nanometer dimensions (e.g., from about 1 nm to about 995 nanometers in diameter, or more typically, from about 2 to about 700 nm in diameter, or from about 4 to about 600 nanometers in diameter. Naturally-occurring and synthetic phophophospholipid-apolipoprotein particles are described, for example, in Pownall et al. (1978) Biochemistry 17: 1183-1188; Pownall et al. (1981) Biochemistry 20: 6630-6635; Jonas et al. (1984) J. Biol. Chem. 259: 6369-6375; Jonas et al. (1989) J. Biol. Chem. 264: 4818-4824; Jonas et al. (1993) J. Biol. Chem. 268: 1596-1602; Leroy et al. (1993) J. Biol. Chem. 268: 4798-4805; Tricerri et al. (2000) Biochemistry 39: 14682-14691; Segall et al. (2002) J. Lipid Res. 43: 1688-1700; Manchekar et al. (2004) J. Biol. Chem. 279: 39757-39766; Pearson et al. (2005) J. Biol. Chem. 280: 38576-38582, all incorporated by reference herein in their entireties.

Other terms used in the fields of recombinant nucleic acid technology and molecular and cell biology as used herein will be generally understood by one of ordinary skill in the applicable arts.

### IVPS Systems

The invention uses in vitro protein synthesis systems such as those known in the art, which can include cell extracts of prokaryotic or eukaryotic cells. The cell extracts can be from cells that are mutated in one or more genes, such as, for example, nuclease-encoding genes or protease-encoding genes, or can be cells engineered to express or overexpress one or more endogenous or exogenous genes, such as, for example, genes encoding tRNAs, polymerases, enzyme inhibitors, etc. The cell extracts can be supplemented with proteins or other molecules that can prevent template degradation, enhance transcription or translation, etc.

Nonlimiting examples of in vitro protein synthesis (IVPS) systems that can be used in the methods and compositions of the invention include but are not limited to those described in, for example, U.S. Pat. No. 5,478,730, to Alakhov et al., entitled “Method of preparing polypeptides in cell-free translation system”; U.S. Pat. Nos. 5,665,563; 5,492,817; and 5,324,637, to Beckler et al., entitled “Coupled transcription and translation in eukaryotic cell-free extract”; U.S. Pat. No. 6,337,191 to Swartz et al., entitled “In vitro Protein Synthesis using Glycolytic Intermediates as an Energy Source”; U.S. Pat. No. 6,518,058 to Biryukov et al., “Method of preparing polypeptides in cell-free system and device for its realization”; U.S. Pat. No. 6,670,173, to Schels et al., entitled “Bioreaction module for biochemical reactions”; U.S. Pat. No. 6,783,957 to Biryukov et al., entitled “Method for synthesis of polypeptides in cell-free systems”; United States Patent Application 2002/0168706 to Chatteijee et al., published Nov. 14, 2002, entitled “Improved in vitro synthesis system”; U.S. Pat. No. 6,168,931 to Swartz et al., issued Jan. 8, 2002, entitled “In vitro macromolecule biosynthesis methods using exogenous amino acids and a novel ATP regeneration system”; U.S. Pat. No. 6,548,276 to Swartz et al., issued Apr. 15, 2003, entitled “Enhanced in vitro synthesis of active proteins containing disulfide bonds”; United States Patent Application 2004/0110135 to Nemetz et al., published Jun. 10, 2004, entitled “Method for producing linear DNA fragments for the in vitro expression of proteins”; United States Patent Application 2004/0209321 to Swartz et al., published Oct. 21, 2004, entitled “Methods of in vitro protein synthesis”; United States Patent Application 2004/0214292 to Motoda et al., published Oct. 28, 2004, entitled “Method of producing template DNA and method of producing protein in cell-free protein synthesis system using the same”; United States Patent Application 2004/0259081 to Watzele et al., published Dec. 23, 2004, entitled “Method for protein expression starting from stabilized linear short DNA in cell-free in vitro transcription/translation systems with exonuclease-containing lysates or in a cellular system containing exonucleases”; United States Patent Applications 2005/0009013, published Jan. 13, 2005, and 2005/0032078, published Feb. 10, 2005, both to Rothschild et al. and both entitled “Methods for the detection, analysis and isolation of nascent proteins”; United States Patent Application 2005/0032086 to Sakanyan et al., published Feb. 10, 2005, entitled “Methods of RNA and protein synthesis”; Published PCT patent application WO 00/55353 to Swartz et al., published Mar. 15, 2000, entitled “In vitro macromolecule biosynthesis methods using exogenous amino acids and a novel ATP regeneration system”. All of these patents and patent applications are hereby incorporated by reference in their entireties.

The preparation of cell extracts that support the synthesis of proteins in vitro from purified mRNA transcripts, or from mRNA transcribed from DNA during the in vitro synthesis reaction are well known in the art. To synthesize a protein under investigation, a translation extract is “programmed” with an mRNA corresponding to the gene and protein under investigation. The mRNA can be produced from DNA, or the mRNA can be added exogenously in purified form. The RNA can be prepared synthetically from cloned DNA using RNA polymerases in an in vitro reaction.

Both prokaryotic cells and eukaryotic cells can be used for protein and/or nucleic acid synthesis according to the invention (see, e.g., Pelham et al, European Journal of Biochemistry, 67: 247, 1976). Prokaryotic systems can be used for simultaneous or “coupled” transcription and translation. The cell extracts used for IVTT contain the components necessary both for transcription (to produce mRNA) and for translation (to synthesize protein) in a single system. In such a system, the input template nucleic acid molecule is DNA.

As demonstrated by the Examples provided herein, the cell-free extracts used in the methods can be prokaryotic or eukaryotic extracts. Eukaryotic in vitro protein synthesis (IVPS) extracts include without limitation rabbit reticulocyte lysates, wheat germ lysates, Drosophila embryo extracts, scallop lysates (Storch et al. J. Comparative Physiology B, 173:611-620, 2003), extracts from mouse brain (Campagnoni et al., J Neurochem. 28:589-596, 1977; Gilbert et al. J Neurochem. 23:811-818, 1974), and chick brain (Liu et al. Transactions of the Illinois State Academy of Science, Volume 68, 1975). A eukaryotic extract for IVPS can be an extract of cultured cells. Cultured cells can be of any type. As nonlimiting examples, HeLa or CHO cell extracts can be used for in vitro translation systems.

Eukaryotic extracts, optionally with added enzymes, substrates, and/or cofactors, can be used for translating proteins with post-translational modifications. Enzymes, substrates and/or cofactors for post-translational modification can also be added to prokaryotic extracts for IVPS. Cell-free extracts can be made using detergent, which is added to cells or cell lysate prior to centrifuging the lysate to make extract, as described in US Patent Application Publication No.2006/0110788 (application Ser. No. 11/240,651), herein incorporated by reference in its entirety for all disclosure of methods and compositions for in vitro protein synthesis systems. For example, nonionic or zwitterionic detergents can be used in the preparation of translation extracts, at concentrations at or slightly above the CMC.

Prokaryotic extracts can be from any prokaryotic cells, including, without limitation, gram negative and gram positive bacteria, including Escherichia sp. (e.g., E. coli), Klebsiella sp., Streptomyces sp., Streptocococcus sp., Shigella sp., Staphylococcus sp., Erwinia sp., Klebsiella sp., Bacillus sp. (e.g., B. cereus, B. subtilis and B. megaterium), Serratia sp., Pseudomonas sp. (e.g., P. aeruginosa and P. syringae), Salmonella sp. (e.g., S. typhi and S. typhimurium), and Rhodobacter sp. Bacterial strains and serotypes suitable for the invention can include E. coli serotypes K, B, C, and W. A typical prokaryotic cell extract is made from E. coli strain K-12. Cell extracts can be made from bacterial strains mutated to lack a nuclease or protease activity, or to lack the activity of one or more proteins that can interfere with purification or detection of translated proteins (see U.S. Patent Publication No. US2005/0136449, herein incorporated by reference in its entirety).

IVPS systems can allow simultaneous and rapid expression of various proteins in a multiplexed configuration, for example in an array format, and can be used for screening of multiple proteins. IVTT systems that use DNA templates can provide increased efficiency in these formats by eliminating the need to separately synthesize and subsequently purify RNA transcripts. In addition, various kinds of unnatural amino acids can be efficiently incorporated into proteins for specific purposes using IVPS systems (see, for example, Noren et al., Science 244:182-188, 1989).

In certain aspects, the cellular extract or an IVPS system that uses the extract, additionally includes at least one other component of any of the components in U.S. Pub. Pat. App. No. 2002/0168706, incorporated herein in its entirety. For example, the cellular extract can include one inhibitor of at least one enzyme, e.g., an enzyme selected from the group consisting of a nuclease, a phosphatase and a polymerase; and optionally the extract can be modified from a native or wild type extract to exhibit reduced activity of at least one enzyme, e.g., an enzyme selected from the group consisting of a nuclease, a phosphatase and a polymerase; and at least two energy sources that supply energy for protein and/or nucleic acid synthesis. In certain aspects the extract includes the Gam protein.

Enzymes, substrates and/or cofactors for post-translational modification can optionally be added to prokaryotic or eukaryotic extracts for IVPS, or may be present in a eukaryotic cell extract.

In addition to a cell extract, an IVPS typically includes at least one amino acid. Typically, an IVPS comprises a cell extract, at least one amino acid, and at least one energy source that supports translation. Where the in vitro translation system is a transcription/translation system, a polymerase is also preferably added. Where the in vitro translation system is a transcription/translation system, a polymerase is also preferably added. In vitro protein synthesis systems, including their manufacture and methods of use, are well known in the art. In exemplary embodiments, at least two amino acids and at least one compound that provides energy for translation is added to a cell extract to provide an IVPS system. In some exemplary embodiments, an IVPS comprises a cell extract, the twenty naturally-occurring amino acids, and at least one compound that provides energy for translation. In some preferred embodiments, an IVPS includes at least two compounds that serve as energy sources for translation, at least one of which can be a glycolytic intermediate. At least one of the amino acids provided in an IVPS system can optionally be labeled, for example, one or more amino acids can be radiolabeled for detection of a translated protein that incorporates the labeled amino acid. In some embodiments, a feeding solution that comprises one or more additional energy sources and additional amino acids is added after an initial incubation of the IVPS. Feeding solutions for IVPS systems and their use are described in U.S. Patent Application Publication No. 2006/0110788, incorporated by reference herein.

Some examples of IVPS systems and other related embodiments are disclosed in U.S. Patent Application Publication No. 2002/0168706, “Improved In vitro Synthesis Systems” filed Mar. 7, 2002; U.S. Patent Application Publication No. 2005/0136449, “Compositions and Methods for Synthesizing, Purifying, and Detecting Biomolecules” filed Oct. 1, 2004; U.S. Patent Application Publication No. 2006/0084136, “Production of Fusion Proteins by Cell-Free Protein Synthesis” filed Jul. 14, 2005; U.S. Patent Application Publication No. 2006/0110788, “Feeding Buffers, Systems, and Methods for In vitro Synthesis” filed Oct. 1, 2005; U.S. Patent Application Publication No. 2006/0110788, “Feeding Buffers, Systems, and Methods for In vitro Synthesis” filed Oct. 1, 2005; and U.S Patent Application Publication No. 2006/0211083, filed Jan. 20, 2006, “Products and Processes for In vitro Synthesis of Biomolecules” the disclosures of which applications are incorporated by reference herein in their entireties.

In some embodiments, the invention uses Invitrogen's EXPRESSWAY™ in vitro translation systems (Invitrogen, Carlsbad, Calif.) that include a cell-free S30 extract and a translation buffer. The S30 extract contains the majority of soluble translational components including initiation, elongation and termination factors, ribosomes and tRNAs from intact cells. The translation buffer contains amino acids, energy sources such as ATP and GTP, energy regenerating components such as phosphoenol pyruvate/pyruvate kinase, acetyl phosphate/acetate kinase or creatine phosphate/creatine kinase and a variety of other important co-factors (Zubay, Ann. Rev. Genet. 7:267-87, 1973; Pelham and Jackson, Eur J Biochem. 67:247, 1976; and Erickson and Blobel, Methods Enzymol. 96;38-50, 1983). The reaction buffer, methionine, T7 Enzyme Mix, and DNA template of interest, operably linked to a T7 promoter, are mixed with the E. coli extract. As the DNA template is transcribed, the 5′ end of the mRNA becomes bound by ribosomes and undergoes translation to synthesis the encoded protein.

### Apolipoproteins

The invention includes methods and compositions in which one or more apolipoproteins is present in an in vitro protein synthesis system. An apolipoprotein can be present in a cell extract when a template encoding a POI is added, or can be added during the synthesis reaction, or an apolipoprotein can be translated from a nucleic acid construct added to the IVPS system.

Apolipoproteins are proteins that bind and transport lipids in the circulatory system of animals. Sequence homology studies across species and structural analysis and predictions indicate that apolipoproteins have similar structure, which includes several amphipathic helices. Accordingly, variant apolipoproteins or engineered apolipoproteins provided herein typically include at least one and can include 2, 3, 4, or more amphipathic helices, typically that includes the sequence of an amphipatihic helix of a wild-type apolipoprotein, or a conservative amino acid substitution thereof. Furthermore, a variant or engineered apolipoprotein used in the methods and compositions of the invention typically retains the ability to bind lipids.

As used herein, the term “apolipoprotein” is used broadly to mean proteins that bind lipids, and are soluble in aqueous solution in both their free and lipid-bound forms. Apolipoproteins of the invention have at least one helical domain that preferably forms, or is predicted to form, an amphipathic helix. Apolipoproteins used in the methods and compositions of the invention preferably are either: naturally-occurring apolipoproteins, which can be of any species origin, sequence variants of naturally-occurring apolipoproteins, as described in more detail below, or an engineered proteins having at least one helical domain that has at least 90% homology to at least one helical domain of a naturally-occurring apolipoprotein. Apolipoproteins used in the methods and compositions of the present invention have the property of when present in an in vitro protein synthesis system (an in vitro translation system), increasing the soluble yield of a membrane protein by at least 10%, where the soluble yield is calculated as either: the amount of soluble protein synthesized, or the percentage of soluble protein to total protein synthesized.

Apolipoproteins used in the methods and compositions of the invention include apolipoprotein variants, including proteins having at least 10, 15, 20, 25, 50, 75, 100, 150, or 200 consecutive amino acids that have at least 50, 60, 70, 75, 80, 85, 90, 95, 96, 97, 98, or 99% sequence identity to a wild-type apolipoprotein of any species, in which the variant, when present in an IVPS system, increases the solubility of at least one protein translated in the IVPS system by at least 10%. In certain aspects, the soluble protein produced in an IVPS system is increased by at least 15%, 20%, or 25%, or is increased in a detectable manner, over the same protein produced in the IVPS system in the absence of the apolipoprotein or variant thereof. Apolipoprotein variants can have one or more sequence deletions or insertions with respect to naturally-occurring apolipoproteins. As nonlimiting examples, tag sequences can be added, or non-helical domains deleted in some apolipoprotein variants.

A variant apolipoprotein, in certain aspects, is a variant of a wild-type mammalian apolipoprotein, especially a variant of Apolipoprotein A-I (Apo A-I), Apolipoprotein A-II (Apo A-II), Apolipoprotein A-IV (Apo A-IV), Apolipoprotein A-V (Apo A-V), Apolipoprotein B-100 (Apo B-100), Apolipoprotein B-48 (Apo B-48), Apolipoprotein C-I (Apo C-I), Apolipoprotein C-II (Apo C-II), Apolipoprotein C-III (Apo C-III), Apolipoprotein D (Apo D), Apolipoprotein E (Apo E), Apolipoprotein H (Apo H), or Lipoprotein (a) (Lp(a)).

Some apolipoproteins, called exchangeable apolipoproteins, reversibly bind lipid, and have stable conformations when bound to lipid and when not bound to lipid. The exchangeable apoplipoproteins are typically less than about 50 kDa in size, and share structural similarity based on a variable number of amphipathic alpha helical domains that are thought to bind the surface of lipoprotein particles (Segrest et al. J. Lipid Res. 33: 141-166 (1992); Pearson et al. J. Biol. Chem. 280, 38576-38582 (2005); Boguski et al. Proc. Natl. Acad. Sci. U.S.A. 83: 8457-8461 (1985)). Exchangeable apolipoproteins include, without limitation, Apolipoprotein A-I, Apolipoprotein A-II, Apolipoprotein A-IV, Apolipoprotein A-V, Apolipoprotein C-I, Apolipoprotein C-II, Apolipoprotein C-III, Apolipoprotein E, and Apoliphorin III.

The apolipoproteins used in the compositions and methods of the invention can be of any animal origin, or based on the sequence of apolipoproteins of any animal species. In some embodiments, the apolipoprotein used in the method of the invention is a mammalian apolipoprotein, is an apolipoprotein variant that has one or more sequences derived from a sequence of one or more mammalian apolipoproteins, such as, for example, Apolipoprotein A-I, Apolipoprotein A-II, Apolipoprotein A-IV, Apolipoprotein A-V, Apolipoprotein B-100, Apolipoprotein B-48, Apolipoprotein C-I, Apolipoprotein C-II, Apolipoprotein C-III, Apolipoprotein D, Apolipoprotein E, Apolipoprotein H, or Lipoprotein (a). The designations of these apolipoproteins used herein may originate from their identification in one or more species; in many cases, the names designate human proteins. For example, the sequences of human apolipoproteins include, without limitation: gi 37499465 (human apolipoprotein A1, SEQ ID NO:1), human proapolipoprotein A1 (SEQ ID NO:2); human apolipoprotein A-II (gi 296633, SEQ ID NO:3), human apolipoprotein A-IV (gi 178759, SEQ ID NO:4); human apolipoprotein A-V (gi 60391728, SEQ ID NO:5), Apolipoprotein B-100, (gi 114014, SEQ ID NO:6); Apolipoprotein B-48 (gi 178732, SEQ ID NO:7); Apolipoprotein C-I (gi 30583123, SEQ ID NO:8); Apolipoprotein C-II (gi 37499469; SEQ ID NO:9); Apolipoprotein C-III (gi 521205, SEQ ID NO:10); Apolipoprotein D (gi5466584, SEQ ID NO:11; gi 1246096, SEQ ID. NO:12); Apolipoprotein E (gi 178853, SEQ ID NO:13); Apolipoprotein H (gi 178857, SEQ ID NO:14); and Apolipoprotein Lp(a) (gi 5031885, SEQ ID NO:15); and their variants having at least 10, 15, 20, 25, 50, 75, 100, 150, or 200 consecutive amino acids that have at least 50, 60, 70, 75, 80, 85, 90, 95, 96, 97, 98, or 99% sequence identity to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, and SEQ ID NO:18 are apolipoproteins that are included in the methods and compositions of the invention.

The designations of Apolipoprotein A-I, Apolipoprotein A-II, Apolipoprotein A-IV, Apolipoprotein A-V, Apolipoprotein B-100, Apolipoprotein B-48, Apolipoprotein C-I, Apolipoprotein C-II, Apolipoprotein C-III, Apolipoprotein D, Apolipoprotein E, Apolipoprotein H, or Lipoprotein (a) however are used herein to also refer to analogues of these proteins in species other than homo sapiens (including but not limited to species of mammal, fish, bird, marsupial, reptile and amphibian). The analogues of the proteins referenced herein by their assigned name for homo sapiens proteins are thus included as apolipoproteins of the invention. Such apolipoproteins and apolipoprotein variants of the invention from species other than homo sapiens may or may not have the same name in other species.

As nonlimiting examples, an Apolipoprotein A-I of any of: rat (gi 6978515), mouse (gi 2145141), golden hamster (gi 4063843), Atlantic salmon (gi 64356), zebrafish (gi 18858281), duck (gi 627301), pufferfish (gi 57157761), orangutan (gi 23379768), chimpanzee (gi 23379764), gorilla (gi 23379766), pig (gi 47523850), baboon (gi 86653), rabbit (gi 71790), or sequence variants thereof, can be used. As nonlimiting examples, an Apolipoprotein A-II of any of: rat (gi 202948), mouse (gi 7304897), macaque (gi 38049), cow (gi 6225059), horse (gi 47115663), or sequence variants thereof, can be used. As nonlimiting examples, an Apolipoprotein A-IV of any of: rat (gi 8392909), mouse (gi 6680702), chicken (gi 45384392), baboon (gi 510276), pig (gi 47523830), chimpanzee (gi 601801), or sequence variants thereof, can be used. As nonlimiting examples, an Apolipoprotein A-V of any of: rat (gi 18034777), mouse (gi 31560003), cow (gi 76635264), or dog (gi 57086253), or sequence variants thereof, can be used.

As nonlimiting examples, an Apolipoprotein B of any of rat (gi 61098031), chicken (gi 114013), rabbit (gi 114015), lemur (gi 31558958), pig (gi 951375), macaque (gi 930126), squirrel (gi 31558956), hedgehog (gi 31558952), or sequence variants thereof, can be used.

As nonlimiting examples, an Apolipoprotein C-I of any of: rat (gi 6978521), mouse (gi 6680704), macaque (gi 114017), rabbit (gi 416626), or sequence variants thereof, can be used. As nonlimiting examples, an Apolipoprotein C-II of any of mouse (gi 6753100), dog (gi 50979236), macaque (gi 342077), guinea pig (gi 191239), cow (gi 114019), pufferfish (gi 74096407), or sequence variants thereof, can be used. As nonlimiting examples, an Apolipoprotein C-III of any of: rat (gi 8392912), mouse (gi 15421856), dog (gi 50979230), pig (gi 50657386), cow (gi 47564119), or sequence variants thereof, can be used.

As nonlimiting examples, an Apolipoprotein D of any of: rat (gi 287650), mouse (gi 75677437), chicken (gi 58696426), guinea pig (gi 1110553), or deer (gi 82469911), or sequence variants thereof, can be used.

As nonlimiting examples, an Apolipoprotein E of any of: rat (gi 20301954), mouse (gi 6753102), chimpanzee (gi 57113897), rhesus monkey (gi 3913070), baboon (gi 176569), pig (gi 311233), cow (gi 312893), or sequence variants thereof, can be used.

As nonlimiting examples, an Apolipoprotein H of any of: rat (gi 56971279), mouse (gi 94400779), woodchuck (gi 92111519), dog (gi 54792721), cow (gi 27806741), or sequence variants thereof, can be used.

In some embodiments, an apolipoprotein used in the method of the invention is an insect apolipoprotein, or has sequences derived from the sequences of an insect apolipoprotein, such as, for example, Apoliphorin I, Apoliphorin II, or Apoliphorin III. Such proteins can be of any species, such as for example, Drospophila species, Manduca species, Locusta species, Lethocerus species, Ostrinia species, Bombyx species, and also their analogues in other insect or in non-insect species. For example, Apolipophorin (gi 2498144, SEQ ID NO:16), Apolipophorin II (gi 2746729, SEQ ID NO:17); Apolipophorin III (gi 159481, SEQ ID NO:18);, and apolipoprotein variants having at least 10, 15, 20, 25, 50, 75, 100, 150, or 200 consecutive amino acids that have at least 50, 60, 70, 75, 80, 85, 90, 95, 96, 97, 98, or 99% sequence identity to SEQ ID NO: 16, SEQ ID NO: 17, and SEQ ID NO: 18 are apolipoproteins that can be used in the compositions and methods of the invention.

Apolipoproteins that can be present in an IVPS system of the invention include, without limitation, Apolipoprotein A-I, Apolipoprotein A-II, Apolipoprotein A-IV, Apolipoprotein A-V, Apolipoprotein B-100, Apolipoprotein B-48, Apolipoprotein C-I, Apolipoprotein C-II, Apolipoprotein C-III, Apolipoprotein D, Apolipoprotein E, Apolipoprotein H, Lipoprotein (a), Apoliphorin I, Apoliphorin II, or Apoliphorin III analogues of any species, including variants of analogues of any species.

In some exemplary embodiments, an apolipoprotein present in an IVPS system is an exchangeable apolipoprotein, such as, for example, Apolipoprotein A-I, Apolipoprotein A-II, Apolipoprotein A-IV, Apolipoprotein A-V, Apolipoprotein C-I, Apolipoprotein C-II, Apolipoprotein C-III, Apolipoprotein E, or Apoliphorin III.

In some embodiments, an apolipoprotein used in the compositions and methods of the invention has at least 70% identity to at least 20 consecutive or contiguous amino acids of an apolipoprotein, such as but not limited to, Apolipoprotein A-I, Apolipoprotein A-II, Apolipoprotein A-IV, Apolipoprotein A-V, Apolipoprotein B-100, Apolipoprotein B-48, Apolipoprotein C-I, Apolipoprotein C-II, Apolipoprotein C-III, Apolipoprotein D, Apolipoprotein E, Apolipoprotein H, Lipoprotein (a), Apoliphorin I, Apoliphorin II, or Apoliphorin III of any species. An apolipoprotein has, in preferred embodiments, at least 70% identity to an apolipoprotein over a continuous sequence of at least 20 amino acids, over a continuous sequence of at least 30 amino acids, over a continuous sequence of at least 40 amino acids, over a continuous sequence of at least 50 amino acids, over a continuous sequence of at least 60 amino acids, over a continuous sequence of at least 70 amino acids, over a continuous sequence of at least 80 amino acids, over a continuous sequence of at least 90 amino acids, or over a continuous sequence of at least 100 amino acids of the apolipoprotein. In some preferred embodiments, an apolipoprotein when present in an IVPS system improves the solubility of at least one protein synthesized in the IVPS system, and has at least 70% identity to an apolipoprotein over a continuous sequence of at least 20 amino acids, over a continuous sequence of at least 30 amino acids, over a continuous sequence of at least 40 amino acids, over a continuous sequence of at least 50 amino acids, over a continuous sequence of at least 60 amino acids, over a continuous sequence of at least 70 amino acids, over a continuous sequence of at least 80 amino acids, over a continuous sequence of at least 90 amino acids, or over a continuous sequence of at least 100 amino acids of the apolipoprotein. In some embodiments, an apolipoprotein used in the methods and compositions of the invention when present in an IVPS system improves the solubility of at least one protein synthesized in the IVPS system, and has at least 70%, at least 80%, at least 90%, at least 95%, or at least 99% identity to an apolipoprotein of any species over a continuous sequence of at least 20 amino acids.

In some embodiments, an apolipoprotein used in the compositions and methods of the invention has at least 70% at least 80%, at least 90%, at least 95%, or at least 99% identity to an exchangeable apolipoprotein, such as but not limited to, Apolipoprotein A-I, Apolipoprotein A-II, Apolipoprotein A-IV, Apolipoprotein C-I, Apolipoprotein C-II, Apolipoprotein C-III, Apolipoprotein E, or Apoliphorin III of any species over a continuous sequence of at least 20 amino acids, at least 30 amino acids, at least 40 amino acids, at least 50 amino acids, at least 60 amino acids, at least 70 amino acids, at least 80 amino acids, or at least 100 amino acids. In some embodiments, an apolipoprotein used in the methods and compositions of the invention when present in an IVPS system improves the solubility of at least one protein synthesized in the IVPS system, and has at least 70% identity to an apolipoprotein of any species over a continuous sequence of at least 20 amino acids, at least 30 amino acids, at least 40 amino acids, at least 50 amino acids, at least 60 amino acids, at least 70 amino acids, at least 80 amino acids, or at least 100 amino acids.

In some embodiments, an apolipoprotein is a mammalian apolipoprotein or has at least 70%, at least 80%, at least 90%, at least 95%, or at least 99% identity to a mammalian apolipoprotein such as, but not limited to, Apolipoprotein A-I, Apolipoprotein A-II, Apolipoprotein A-IV, Apolipoprotein A-V, Apolipoprotein B-100, Apolipoprotein B-48, Apolipoprotein C-I, Apolipoprotein C-II, Apolipoprotein C-III, Apolipoprotein D, Apolipoprotein E, Apolipoprotein H, or Lipoprotein (a) over a continuous sequence of at least 20 amino acids, at least 30 amino acids, at least 40 amino acids, at least 50 amino acids, at least 60 amino acids, at least 70 amino acids, at least 80 amino acids, or at least 100 amino acids.

In some embodiments, an apolipoprotein is an insect apolipoprotein such as Apoliphorin I, Apoliphorin II, or Apoliphorin III, or has at least 70%, at least 80%, at least 90%, at least 95%, or at least 99% identity to an insect Apoliphorin I, Apoliphorin II, or Apoliphorin III over a continuous sequence of at least 20 amino acids, at least 30 amino acids, at least 40 amino acids, at least 50 amino acids, at least 60 amino acids, at least 70 amino acids, at least 80 amino acids, or at least 100 amino acids.

In some exemplary embodiments, an apolipoprotein used in the methods and compositions of the invention is a wild-type exchangeable apolipoprotein or a variant thereof having at least 90% sequence identity to at least 100 contiguous amino acids of the wild-type exchangeable apolipoprotein, and capable of increasing the soluble protein production of a bacterial EmrE protein or a human GABA receptor protein in an in vitro protein synthesis reaction by at least 10%. In some embodiments, an apolipoprotein used in the methods and compositions of the invention is Apolipoprotein A-I, Apolipoprotein A-II, Apolipoprotein A-IV, Apolipoprotein A-V, Apolipoprotein C-I, Apolipoprotein C-II, Apolipoprotein C-III, Apolipoprotein E, or Apoliphorin III, or a variant of any of these having at least 90% sequence identity to at least 100 contiguous amino acids of the wild-type exchangeable apolipoprotein, and capable of increasing the soluble protein production of a bacterial EmrE protein or a human GABA protein in an in vitro protein synthesis reaction by at least 10%.

In an exemplary embodiment, an apolipoprotein used in the methods and compositions of the invention is Apolipoprotein A-I or a variant of Apolipoprotein A-I having at least 90% sequence identity to at least 100 contiguous amino acids of wild-type Apolipoprotein A-I, and having the ability to increase soluble protein production of the bacterial EmrE protein or the human GABA protein in an in vitro protein synthesis reaction by at least 10%.

The apolipoproteins of the invention also include engineered apolipoproteins having at least 90% amino acid sequence identity with at least 10 residues of a helical domain of a naturally-occurring apolipoprotein. The invention includes engineered apolipoproteins (“membrane scaffold proteins”) disclosed in US Patent Application Publication 2005/0182243, herein incorporated by reference, including, but not limited to: histidine tagged MSP1 (SEQ ID NO: 19); MSP1 (SEQ ID NO:20); MSP2 (his tagged) (SEQ ID NO:21); MSP2 (his tagged, long linker) (SEQ ID NO:22); MSP1D5D6 (SEQ ID NO:23); MSP1D6D7 (SEQ ID NO:24); MAP1T4 (SEQ ID NO:25); MSP1T5 (SEQ ID NO:26); MSP1T6 (SEQ ID NO:27); MSP1N1 (SEQ ID NO:28); MSP1E3TEV (SEQ ID NO:29); MSP1E3D1 (SEQ ID NO:30); HisTEV-MSP2 (SEQ ID NO:31); MSP2N1 (SEQ ID NO:32); MSP2N2 (SEQ ID NO:33); MSP2N3 (SEQ ID NO:34); MSP2N4 (SEQ ID NO:35); MSP2N5 (SEQ ID NO:36); MSP2N6 (SEQ ID NO:37); MSP2CPR (SEQ ID NO:38); His-TEV-MSP1T2-GT (SEQ ID NO:39); MSP1RC12′(SEQ ID NO:40); MSP1K90C (SEQ ID NO:41); and MSP1K152C (SEQ ID NO:42).

The apoplipoproteins used in the methods and compositions of the invention can be from any source, for example, isolated from organisms or tissue, including blood, plasma, or serum, isolated from cell culture, or expressed recombinantly prior to be added to the in vitro synthesis system. Preferably, an apolipoprotein is at least partially purified prior its addition to an in vitro synthesis system.

The amino acid sequence of an apolipoprotein used in the methods and compositions of the invention can be modified with respect to the sequence of a wild-type apolipoprotein, having one or more deletions, additional amino acids, or amino acid substitutions with respect to a wild-type sequence, while having the property of enhancing the yield of protein in soluble form made in an in vitro protein synthesis reaction when the apolipoprotein is present in the in vitro protein synthesis reaction.

For example, an apolipoprotein used in the methods or compositions of the invention can have an N-terminal or C-terminal truncation, or can have one or more internal deletions or insertions with respect to a wild-type apolipoprotein sequence. An apolipoprotein used in the methods and compositions of the invention can be a multimer of an apolipoprotein or a portion thereof, for example, two or more copies of an apolipoprotein, or a variant or portion thereof, joined by a linker. An apolipoprotein used in the methods and compositions of the invention can be a chimeric apolipoprotein, comprising sequences of two different apolipoproteins (or variants thereof). Furthermore, the apolipoprotein can be bound to a peptide or another protein sequence, as part of a fusion protein. The peptide sequence can be a purification and/or detection tag, for example.

In some embodiments of the invention, apolipoproteins used in an IVPS include membrane scaffold proteins (MSPs) based on the sequence of Apolipoprotein A-1 disclosed in U.S. Pat. No. 7,048,949; U.S. Patent Application Publication No. 2005/0182243 A1, 2005/0152984 A1, 2004/0053384 A1, and 2006/0088524 A1, all incorporated by reference herein in their entireties.

The apolipoprotein provided herein can be bound to a lipid or can be a lipid free apolipoprotein. For example, an apolipoprotein can be isolated from an organism (such as from blood or plasma), from tissue culture cells or media, or from bacterial cells engineered to express a recombinant apolipoprotein. The isolated apolipoprotein can be bound to lipid using methods known in the art (see, for example, Pownall et al. (1978) Biochemistry 17: 1183-1188; Pownall et al. (1981) Biochemistry 20: 6630-6635; Jonas et al. (1984) J. Biol. Chem. 259: 6369-6375; Jonas et al. (1989) J. Biol. Chem. 264: 4818-4824; Jonas et al. (1993) J. Biol. Chem. 268: 1596-1602; Tricerri et al. (2000) Biochemistry 39: 14682-14691; Segall et al. (2002) J. Lipid Res. 43: 1688-1700; Pearson et al. (2005) J. Biol. Chem. 280: 38576-38582, all incorporated by reference herein in their entireties). In some embodiments of the invention, apolipoproteins can be provided in in vitro protein synthesis systems that also include one or more lipids, such as but not limited to one or more phospholipids. Cholesterol, a cholesterol ester, or one or more other neutral lipids, such as, but not limited to, a sterol ester, a mono-, di-, or triacylglyceride, or an acylglycerol, can optionally also be included. Lipids can be present at a concentration of from about 1 microgram per milliliter to about 20 milligrams per milliliter, or from about 5 micrograms per milliliter to about 10 milligrams per milliliter, or from about 10 micrograms per milliliter to about 5 milligrams per milliliter. One or more phospholipids can be bound to an apolipoprotein in the in vitro protein synthesis system. In some embodiments of the invention, apolipoproteins are translated using in vitro protein systems that include one or more lipids, such as but not limited to one or more phospholipids. The apolipoproteins synthesized in the cell-free system can bind one or more lipids during or following translation.

### Phospholipid-Apolipoprotein Particles

In some embodiments of the invention, apolipoproteins can be present in an in vitro protein synthesis system as phospholipid-apolipoprotein particles in which the particles comprise phospholipids organized into a bilayer disc bound by the apolipoprotein. Some examples of phospholipid-apolipoprotein particles and methods of making phospholipid-apolipoprotein discs (including phospholipid apolipoprotein disc that comprise apolipoprotein variants) are known in the art and described, for example, in Jonas et al. (1984) J. Biol. Chem. 259: 6369-6375; Jonas et al. (1989) J. Biol. Chem. 264: 4818-4824; Jonas et al. (1993) J. Biol. Chem. 268: 1596-1602; U.S. Pat. No. 7,048,949; U.S. Patent Application Publication No. 2005/0182243 A1, 2005/0152984 A1, 2004/0053384 A1, and 2006/0088524 A1, all incorporated by reference herein in their entireties.

Nanoscopic bilayer discs, herein disclosed as phospholipid-apolipoproteins particles, or PAPs, are described in U.S. Pat. No. 7,048,949, U.S. Patent Application Publication Nos. 2005/0182243, 2005/0152984, 2004/0053384, and WO 02/040501, all of which are incorporated by reference in their entireties, and in particular for disclosure of nanoscopic phospholipids bilayer discs, their components, their manufacture, and methods of use. The methods of the invention produce membrane proteins that are inserted into phospholipid-apolipoprotein particles, or nanoscopic phospholipid bilayer discs. A nucleic acid template is added to an in vitro protein synthesis system that comprises a cell extract and a preparation of PAPs; and the in vitro protein synthesis system is incubated to synthesize a membrane protein in soluble form, in which the membrane protein in soluble form is inserted into PAPs.

The present invention includes translation systems and methods comprising phospholipid bilayer particles or discs that include an apolipoprotein. Preferably the apolipoprotein provided as a phospholipid-apolipoprotein has at least one amphipathic helical domain. The apolipoprotein can be, for example, Apolipoprotein A-I, Apolipoprotein A-II, Apolipoprotein A-IV, Apolipoprotein A-V, Apolipoprotein B-100, Apolipoprotein B-48, Apolipoprotein C-I, Apolipoprotein C-II, Apolipoprotein C-III, Apolipoprotein D, Apolipoprotein E, Apolipoprotein H, Lipoprotein (a), Apolipophorin I, Apolipophorin II, or Apolipophorin III or derivatives or variants thereof (for example, chimeric apolipoproteins, C-terminal or N-terminal truncated apolipoproteins, internally deleted apolipoproteins, apolipoproteins comprising additional amino acid sequences or altered amino acid sequences). In preferred embodiments, a phospholipid-apolipoprotein particle in an IVPS of the invention is Apo A-I, Apo A-IV, Apo A-V, Apo C-I, Apo C-II, Apo C-III, Apo-E, or Apolipophorin III, or a variant of any of these. In some embodiments, the length of an amphipathic helical domain of any apolipoprotein can be altered to promote the formation phospholipid-apolipoprotein particles of different desired diameters. This can be advantageous for accommodating multiple proteins within a phospholipid-apolipoprotein particle.

Phospholipids used to form phospholipid-apolipoprotein particles or discs in translation systems can be glycerol or sphingolipid based, and can contain, for example, two saturated fatty acids of from 6 to 20 carbon atoms and a commonly used head group such as, but not limited to, phosphatidyl choline, phosphatidyl ethanolamine and phosphatidyl serine. The head group can be uncharged, positively charged, negatively charged or zwitterionic. The phospholipids can be natural (those which occur in nature) or synthetic (those which do not occur in nature), or mixtures of natural and synthetic. Nonlimiting examples of phospholipids include, without limitation, PC, phosphatidyl choline; PE, phosphatidyl ethanolamine, PI, phosphatidyl inositol; DPPC, dipalmitoyl-phosphatidylcholine; DMPC, dimyristoyl phosphatidyl choline; POPC, 1-palmitoyl-2-oleoyl-phosphatidyl choline; DHPC, dihexanoyl phosphatidyl choline, dipalmitoyl phosphatidyl ethanolamine, dipalmitoyl phosphatidyl inositol; dimyristoyl phosphatidyl ethanolamine; dimyristoyl phosphatidyl inositol; dihexanoyl phosphatidyl ethanolamine; dihexanoyl phosphatidyl inositol; 1-palmitoyl-2-oleoyl-phosphatidyl ethanolamine; or 1-palmitoyl-2-oleoyl-phosphatidyl inositol; among others.

The isolated apolipoprotein and phospholipids can be mixed to assemble into phospholipid-apolipoprotein, for example, as described in the art, including Jonas et al. (1984) J. Biol. Chem. 259: 6369-6375; Jonas et al. (1989) J. Biol. Chem. 264: 4818-4824; Jonas et al. (1993) J. Biol. Chem. 268: 1596-1602; U.S. Pat. No. 7,048,949; U.S. Patent Application Publication No. 2005/0182243 A1, 2005/0152984 A1, 2004/0053384 A1, and 2006/0088524 A1, all incorporated by reference herein in their entireties. The phospholipid-apolipoprotein particles are then added to a cell extract or IVPS system.

In some other aspects of the invention, a nucleic acid construct encoding an apolipoprotein is provided in an IVPS system that includes one or more phospholipids, and an apolipoprotein translated in vitro associates with phospholipid to form a phosphophospholipid-apolipoprotein particles in the IVPS system.

### Recombinational Cloning

Cloning systems that utilize recombination at defined recombination sites, including the GATEWAY® recombination cloning system, vectors, enzymes, and kits available from Invitrogen (Carlsbad, Calif.) have been previously described in U.S. application Ser. No. 09/177,387, filed Oct. 23, 1998; U.S. application Ser. No. 09/517,466, filed Mar. 2, 2000; and U.S. Pat. Nos. 5,888,732 and 6,277,608, all of which are specifically incorporated herein by reference. These systems can be used for cloning MPOI coding sequences and/or apolipoprotein coding sequences into expression vectors for in vitro translation, and multisite GATEWAY® vectors can be used to accommodate multiple open reading frames for simultaneous translation of two or more proteins in a single reaction.

In brief, the GATEWAY® Cloning System utilizes vectors that contain at least one recombination site to clone desired nucleic acid molecules in vivo or in vitro. More specifically, the system utilizes vectors that contain at least two different site-specific recombination sites based on the bacteriophage lambda system (e.g., att1 and att2) that are mutated from the wild-type (att0) sites. Each mutated site has a unique specificity for its cognate partner att site (i.e., its binding partner recombination site) of the same type (for example, attB1 with attP1, or attL1 with attR1) and will not cross-react with recombination sites of the other mutant type or with the wild-type att0 site. Different site specificities allow directional cloning or linkage of desired molecules thus providing desired orientation of the cloned molecules. Nucleic acid fragments flanked by recombination sites are cloned and subcloned using the GATEWAY system by replacing a selectable marker (for example, ccdB) flanked by att sites on the recipient plasmid molecule, sometimes termed the Destination Vector. Desired clones are then selected by transformation of a ccdB sensitive host strain and positive selection for a marker on the recipient molecule. Similar strategies for negative selection (e.g., use of toxic genes) can be used in other organisms such as thymidine kinase (TK) in mammals and insects.

### Methods and Systems for Synthesizing Proteins in Vitro Using Apolipoproteins

The present invention is based on the finding that membrane proteins can insert into phospholipid-apolipoprotein particles (phospholipids bilayer discs) when the membrane proteins are translated in the presence of phospholipid-apolipoprotein particles (PAPs). As illustrated in the Examples provided herein, synthesis of a membrane protein of interest (MPOI) in an in vitro protein synthesis (IVPS) system that contains PAPs results in production an MPOI with enhanced solubility, in which the MPOI is incorporated into PAPs.

In a further discovery the inventors have found that membrane proteins can be translated in the presence of an apolipoprotein that is not part of a PAP, in which the MPOI translated in the presence of an apolipoprotein has enhanced solubility with respect to the same MPOI translated in vitro in the absence of the apolipoprotein. The invention thus includes in vitro synthesis methods and systems for translating proteins in the presence of an apolipoprotein. The invention includes in vitro synthesis methods and systems for translating proteins in the presence of an apolipoprotein in which the apolipoprotein in the IVPS system is not provided in a PAP. The invention also includes in vitro synthesis methods and systems for translating proteins in the presence of an apolipoprotein in which exogenous phospholipids are not present in the IVPS system.

Yet other features of the invention are based on the finding that an apolipoprotein can be translated in the same IVPS system in which an MPOI is translated, and when both the MPOI and the apolipoprotein are synthesized in the same IVPS reaction, the MPOI has enhanced solubility with respect to its solubility when synthesized in an IVPS reaction that does not contain an apolipoprotein or does not include an apolipoprotein template.

In one aspect, then, the invention provides a method of synthesizing a protein of interest in vitro, comprising: adding a nucleic acid template that encodes a protein of interest to an in vitro protein synthesis system that includes an apolipoprotein and incubating the in vitro protein synthesis system to synthesize the protein of interest. In some preferred embodiments, at least a portion of the protein of interest is synthesized in soluble form.

A protein of interest (“POI”) translated in the IVPS system can be any protein of interest, such as but not limited to: an enzyme, structural protein, carrier protein, binding protein, antibody, hormone, growth factor, receptor, inhibitor, or activator. The Examples provided herein demonstrate the presence of apolipoprotein in an IVPS reaction does not deleteriously affect translation of non-membrane proteins. In some preferred embodiments, a protein of interest translated using the methods of the invention is a membrane protein (“MPOI”), or a protein that in its native state associates with biological membranes, such as, for example, a transmembrane protein, an embedded membrane protein, or a peripheral membrane protein.

In some preferred embodiments, a protein of interest translated using the methods of the invention is a membrane protein, and after incubating the in vitro protein synthesis system a larger amount of the membrane protein of interest (MPOI) is synthesized in soluble form than when the protein is translated in the absence of the apolipoprotein. For example, in preferred embodiments at least 10%, at least 20%, at least 30%, at least 40%, at least 50%, at least 60%, at least 70%, at least 80%, at least 90% or at least 100% more of the MPOI is synthesized in the presence of an apolipoprotein than when there is no apolipoprotein present in the IVPS reaction. In some preferred embodiments, after incubating the IVPS system there is a higher percentage of soluble MPOI to total protein of interest synthesized than when the MPOI is translated in the absence of the apolipoprotein. For example, in preferred embodiments the percentage of soluble MPOI to total MPOI synthesized in an IVPS reaction increases by at least 10%, at least 20%, at least 30%, at least 40%, at least 50%, at least 60%, at least 70%, at least 80%, at least 90% or at least 100% when the MPOI is synthesized in the presence of an apolipoprotein with respect to the percentage of soluble MPOI to total MPOI synthesized when the MPOI is synthesized with no apolipoprotein present in the IVPS reaction.

As described herein, an apolipoprotein provided in an IVPS system is a protein that is either: a naturally-occurring apolipoprotein, which can be of any species origin; a sequence variant of naturally-occurring apolipoprotein; or an engineered protein having at least one helical domain that has at least 90% homology to a helical domain of a naturally-occurring apolipoprotein. Apolipoproteins used in the methods and compositions of the present invention have the property of increasing the soluble yield of a membrane protein by at least 10%, where the soluble yield is calculated as either: the amount of soluble protein synthesized, or the percentage of soluble protein to total protein synthesized, when the apolipoproteins are provided in an IVPS system or translated in an IVPS that is also translating the membrane protein.

An apolipoprotein that is present in an IVPS system can be present at any concentration that permits translation of a MPOI. As general guidelines only, an apolipoprotein can be provided in an IVPS system at concentration of from about 0.5 micrograms per mL to about 2 milligrams per mL, or from about 1 microgram per mL to about 1 mg per mL, or from about 5 micrograms per mL to about 500 micrograms per mL, or from about 10 micrograms per mL to about 250 micrograms per mL. More than one apolipoprotein can be present in a single IVPS reaction.

The one or more apolipoproteins can be added to an IVPS reaction after a nucleic acid template is added to the reaction, but preferably is present in an NPS reaction when a nucleic acid template encoding a POI is added. As used herein, “adding to an IVPS system” means adding to a cell extract prepared for IVPS, to which other components for in vitro synthesis may have already been added, or are yet to be added.

The invention thus includes, in another aspect, a cell extract for in vitro translation that includes at least one apolipoprotein as described herein. Cell extracts for in vitro translation include all those described herein. In some embodiments, the invention includes an IVPS system that includes an apolipoprotein, a cell extract, and a chemical energy source. In some embodiments, the invention includes an IVPS system that includes an apolipoprotein, a cell extract, a chemical energy source, and one or more amino acids. In some embodiments, the invention includes an IVPS system that includes an apolipoprotein, a cell extract, a chemical energy source, one or more amino acids, and a nucleic acid template. The IVPS system can optionally include one or more lipids, detergents, salts, buffering compounds, enzymes, inhibitors, or cofactors.

In some embodiments of the methods of the invention, an apolipoprotein is added to an IVPS system that includes one or more lipids, such as but not limited to one or more phospholipids. In some embodiments of the methods of the invention, an apolipoprotein is added to an IVPS system that includes one or more lipids and the apolipoprotein becomes associated with one or more lipids in the IVPS system. In some embodiments of the methods of the invention, an apolipoprotein is associated with one or more lipids when it is added to an IVPS system. In some embodiments of the invention, an apolipoprotein is added to an IVPS system that includes one or more lipids, or an apolipoprotein is associated with one or more lipids when it is added to an IVPS system, and during incubation of the IVPS system, a synthesized protein of interest become associated with the apolipoprotein and its associated lipid(s) in the IVPS system.

In some embodiments of the methods of the invention, an apolipoprotein added to an IVPS system is added as a phospholipid-apolipoprotein particle (PAP). In some embodiments of the methods of the invention, an apolipoprotein added to an IVPS system is added as a PAP and a MPOI synthesized in the system becomes associated with a PAP.

In a further aspect, therefore, the invention includes a cell extract for translation that includes phospholipid-apolipoprotein particles (PAPs) as described herein, Cell extracts for in vitro translation include all those described herein. In some embodiments, the invention includes an IVPS system that includes PAPs, a cell extract, and a chemical energy source. In some embodiments, the invention includes an IVPS system that includes PAPs, a cell extract, a chemical energy source, and one or more amino acids. In some embodiments, the invention includes an IVPS system that includes PAPs, a cell extract, a chemical energy source, one or more amino acids, and a nucleic acid template. The IVPS system can optionally include one or more lipids, detergents, salts, buffering compounds, enzymes, inhibitors, or cofactors.

Phospholipid-apolipoprotein particles (PAPs) as described in detail above, can be added to or provided in an IVPS system in any concentration that permits in vitro translation, but is preferably added at a concentration that enhances the solubility of a MPOI translated in the IVPS. As general guidelines only, PAPs can be added at concentrations ranging from about 0.5 micrograms per mL to about 2 milligrams per mL, or from about 1 microgram per mL to about 1 mg per mL, or from about 5 micrograms per mL to about 500 micrograms per mL, or from about 10 micrograms per mL to about 250 micrograms per mL, where the concentration given is based on the protein content of the PAPs. More than one type of PAP can be present in a single IVPS reaction, where different PAPs can have different apolipoprotein and/or different phospholipids composition.

The present invention provides efficient systems and methods for synthesizing membrane proteins in a cell-free system in soluble form. The methods include translating membrane proteins in a cell free system that includes phospholipid-apolipoprotein particles.

In some embodiments of the invention, the methods further include isolating the protein of interest from the IVPS mixture. Isolation procedures can be, for example, by means of a peptide tag that is part of the apolipoprotein, or by a peptide tag that is part of the protein of interest.

An apolipoprotein can be provided in an IVPS system by translating the apolipoprotein in the IVPS system that translates the POI. In yet another aspect, therefore, the invention provides a method of synthesizing a protein in vitro, in which the method includes: adding to an in vitro synthesis system a nucleic acid construct that encodes an apolipoprotein and a nucleic acid construct that encodes a protein of interest, and incubating the in vitro protein synthesis system to synthesize an apolipoprotein and a protein of interest. In some preferred embodiments, the protein of interest is synthesized in soluble form. In some preferred embodiments, the protein of interest is a membrane protein.

In some embodiments, an apolipoprotein is provided on a first nucleic acid construct, and a protein of interest is provided on a second nucleic acid construct. In other embodiments of this aspect of the invention, sequences encoding an apolipoprotein and sequences encoding a protein of interest are provided on the same nucleic acid construct. GATEWAY® vectors and cloning systems can optionally be used in making nucleic acid constructs that encode one or both of an apolipoprotein and a protein of interest. In some embodiments, a DNA construct that includes sequences encoding an apolipoprotein and sequences encoding a protein of interest has a first promoter for the apolipoprotein coding sequences a second promoter for the protein of interest coding sequences. In one alternative, a nucleic acid construct that includes sequences encoding an apolipoprotein and sequences encoding a protein of interest include an IRES sequence between the two coding sequences.

In these aspects of the present invention, a nucleic acid construct encoding an apolipoprotein can encode any apolipoprotein as disclosed herein, including a naturally-occurring apolipoprotein, a sequence variant of a naturally-occurring apolipoprotein, or an engineered apolipoprotein having at least one helical domain that has at least 90% homology to a helical domain of a naturally-occurring apolipoprotein. A nucleic acid construct encoding an apolipoprotein can encode an apolipoprotein having an amino acid sequence that is modified with respect to the amino acid sequence of a wild-type apolipoprotein. In some embodiments, a nucleic acid construct encoding an apolipoprotein or apolipoprotein variant encodes a tag sequence fused to the apolipoprotein sequence.

In some preferred embodiments, a protein of interest translated in an IVPS that includes a template encoding an apolipoprotein and a template encoding a membrane protein, and after incubating the in vitro protein synthesis system, a larger amount of the membrane protein of interest (MPOI) is synthesized in soluble form than when the MPOI is translated in the absence of apoliprotein being present or produced in the same reaction. In some preferred embodiments, a protein of interest translated using the methods of the invention is a membrane protein, and after incubating the IVPS system there is a higher percentage of soluble protein of interest to total protein of interest is synthesized than when the protein of interest is translated in the absence of the apolipoprotein being present or translated in the same reaction.

In some embodiments, an in vitro protein synthesis system of the invention that comprises nucleic acid construct(s) encoding a protein of interest and an apolipoprotein comprises one or more lipids, such as but not limited to one or more phospholipids. In some embodiments, methods of the invention that comprise synthesizing a protein of interest in soluble form comprise adding to an in vitro synthesis system that comprises at least one lipid a nucleic acid construct that encodes an apolipoprotein and a nucleic acid construct that encodes a protein of interest and incubating the in vitro protein synthesis system to synthesize an apolipoprotein particle and a protein of interest associated with the phospholipid-apolipoprotein particle.

The invention therefore provides, in a further aspect, an in vitro protein synthesis system that includes a cell extract, a nucleic acid template that encodes an apolipoprotein, and a nucleic acid template that encodes a protein of interest. In certain embodiments, the invention includes an in vitro protein synthesis system that includes a cell extract, a first nucleic acid molecule that encodes an apolipoprotein, and a second nucleic acid molecule that encodes a protein of interest. In other embodiments, an in vitro protein synthesis system that includes a cell extract and a nucleic acid template that encodes an apolipoprotein and a protein of interest. Either or both of the nucleic acid templates can be DNA or RNA.

An apolipoprotein sequence encoded by a nucleic acid construct used in the methods and in vitro synthesis systems of the invention can be the sequence of any apolipoprotein disclosed herein. A construct that encodes an apolipoprotein can also encode an amino acid tag fused in frame with the apolipoprotein sequence. A nucleic acid template that encodes an apolipoprotein can be a DNA template or an RNA template. A nucleic acid template that encodes an apolipoprotein can be bound to a solid support, such as, for example, a bead, matrix, chip, array, membrane, sheet, dish, or plate.

A nucleic acid template that encodes a protein of interest can be a DNA template or an RNA template, and can encode any protein of interest, such as but not limited to: an enzyme, structural protein, carrier protein, hormone, growth factor, inhibitor, or activator. In some preferred embodiments, a protein of interest translated using the methods of the invention is a membrane protein. A construct that encodes a protein of interest can also encode an amino acid tag fused in frame with the protein of interest sequence.

A nucleic acid construct present in an in vitro protein synthesis system of the invention can encode more than one protein of interest. A nucleic acid template that encodes a protein of interest can be bound to a solid support, such as, for example, a bead, matrix, chip, array, membrane, sheet, dish, or plate.

The invention also provides methods for efficient systems and methods for in vitro synthesis of membrane proteins in soluble and readily purifiable form. In these methods, an MPOI is synthesized in an in vitro translation reaction that includes an apolipoprotein, in which the apolipoprotein has a purification tag. Capture of the apolipoprotein using the purification tag leads to the co-isolation of membrane proteins synthesized in vitro in the presence of the apolipoprotein. In embodiments in which the apoliprotein is incorporated into a PAP, capture of the apolipoprotein using the purification tag leads to isolation of PAPs that include the MPOI. The PAPs having incorporated MPOIs can be used for any of a number of assays, and also for structural studies, such as but not limited to NMR or X-ray crystallography.

In another embodiment, a membrane protein of interest (MPOI) can optionally be translated in the presence of an apolipoprotein, in which the MPOI has a protein tag attached for further identification, isolation, tethering, or purification or immobilization of the synthesized protein. In this case, the apolipoprotein can optionally also have a tag.

The present invention further provides methods for in vitro synthesis of POIs, including MPOIs, where the identity of the proteins may be known or unknown, in IVPS reactions that include apolipoproteins (in the context of PAPs or not in PAPs), in which multiple reactions are performed in parallel, for example, in a multiwell plate to obtain multiple solubilized proteins for assays. The proteins can be expressed from vector-driven templates, where the vectors include transcriptional and translational expression sequences located near cloning sites. The vectors can be used to clone libraries of sequences, and can optionally include protein tag sequences that can be translated in frame with the POIs.

In one preferred embodiment, an apolipoprotein of a PAP can include an affinity tag (such as a his tag, glutathione tag, streptavidin tag, etc.) used to tether the PAP containing a MPOI to a solid support, such as but not limited to a microwell surface, a chip surface, a sheet, a membrane, a matrix or bead. MPOIs translated with PAPs can be immobilized to a microwell, chip surface, sheet, membrane, matrix, or bead via their insertion into the tethered PAPs. The PAP can be tethered to the solid support before or after translation of the MPOI in the presence of the PAP.

Thus, the methods of the present invention can be used to make membrane protein arrays or multiwell assay plates, where localized in vitro translation reactions that include PAPs allow for tethering of PAPs having individual MPOIs inserted to specific locations on the array. Such arrays can be used for many types of screens and assays, including but not limited to enzymatic assays, ion channel assays, and drug binding assays. Labeling of MPOIs in the translation reaction, as described below, can be performed for facilitating array assays.

The arrays or multiwell assay plates can be made by in vitro translation reactions that are performed on the array or plate itself For example, each location on an array, or well or a plate, can receive an NPS reaction that includes a cell extract, PAPs, and a nucleic acid template that encodes an MPOI. The PAPs can become tethered to the array via a his, glutathione, streptavidin, or other tag engineered into the apolipoprotein. An MPOI can be a known or unknown protein.

In another embodiment the MPOI can be cloned into a vector that provides a sequence that encodes a tag as an N-terminal or C-terminal amino acid sequence of the protein of interest. The tag can be used for further isolation, tethering, or purification or immobilization of the proteins, which can be translated in the presence of an apolipoprotein that can be provided without associated phospholipids, or in the context of PAPs. The synthesized protein can be captured, for example, to the bottom of a well, or an array locus or well, or to a filter, matrix, or bead, that has been treated or coated with an affinity capture reagent.

The invention also includes methods of translating membrane proteins in an IVPS system that includes an apolipoprotein in which the MPOIs are labeled during translation, such as, for example, with a radiolabel, a heavy isotope label, or a fluorescent label (such as BODIPY® FL fluorophore incorporated at the N-terminus through inclusion of tRNA met (fmet) misaminoacylated with a methionine containing a BODIPY® FL fluorophore at its amino group). Alternatively, MPOIs can be engineered to contain a tag that can bind a label, such as, for example, a fluorescent label (as nonlimiting examples, LUMIO™ tetracysteine sequence motif detection technology can be used (Invitrogen, Carlsbad, Calif.; see for example US 2003/0083373, U.S. Pat. No. 5,932,474, U.S. Pat. No. 6,008,378, U.S. Pat. No. 6,054,271, WO 99/21013, all herein incorporated by reference in their entireties) or PRO-Q® Sapphire 532, 365, or 488 Oligohistidine stain for his-tagged proteins (Invitrogen, Carlsbad, Calif.). The method includes: translating a membrane protein in an in vitro synthesis reaction that includes an apolipoprotein and at least one label that can be incorporated into the synthesized membrane protein, In an alternative embodiment, the method includes: translating a membrane protein in an in vitro synthesis reaction that includes at least one apolipoprotein where the translated membrane protein includes at least one tag that can bind a label. The methods result in the production of labeled or tagged membrane proteins in soluble form. The method in preferred embodiments results in production of a tagged and/or labeled membrane protein membrane protein having enhanced solubility.

In some preferred embodiments of these methods, the apolipoproteins present in the IVPS system are in PAPs. The invention therefore includes: translating a membrane protein in an in vitro synthesis reaction that includes phospholipid-apolipoprotein particles and at least one label that can be incorporated into the synthesized membrane protein to produce a labeled membrane protein. The method includes: translating a membrane protein in an in vitro synthesis reaction that includes phospholipid-apolipoprotein particles and at least one label that can be incorporated into the synthesized membrane protein to produce a labeled membrane protein inserted into phospholipid-apolipoprotein particles. In an alternative embodiment, the method includes: translating a membrane protein in an in vitro synthesis reaction that includes at least one phospholipid-apolipoprotein particle, in which the translated membrane protein includes at least one tag that can bind a label. The method includes: translating a membrane protein in an in vitro synthesis reaction that includes phospholipid-apolipoprotein particles, in which the translated membrane protein includes at least one tag that can bind a label to produce a tagged membrane protein inserted into phospholipid-apolipoprotein particles.

Labeling of a membrane protein that is inserted into PAPs can make possible membrane protein-ligand binding studies, in which ligand binding affects the fluorescence properties of the labeled protein. In related embodiments, the ligand can also be labeled, and fluorescence detection methods such as FRET can be used to assess ligand-membrane protein binding. The present invention thus includes methods of translating a membrane protein in an NPS system that includes PAPs, in which a label or tag that can directly or indirectly bind a label is incorporated into the translated membrane protein.

Labeling of a membrane protein that is inserted into PAPs can also make possible protein-protein interaction studies, including but not limited to membrane protein-protein interaction studies (such as but not limited to receptor dimerization studies) in which protein-protein interaction affects the fluorescence properties of the labeled protein. One or both of the proteins can be labeled.

Assays, including but not limited to assays of ligand binding, ion channel activity, and protein-protein interaction can be conducted on arrays, in which the arrays include PAPs with inserted MPOIs. In this way, assays on membrane proteins can be conducted in a high throughput mode, as laborious and customized purification procedures are obviated.

The present invention also includes methods of incorporating two or more different membrane proteins of interest into a common PAP using in vitro translation methodologies. In these embodiments, the different membrane proteins can be translated in a common in vitro reaction using the same or different nucleic acid template molecules. For example, multi-site GATEWAY® vectors (Invitrogen, Carlsbad, Calif.) can be used to clone at least two open reading frames in the same vector. Labels can be incorporated into the proteins during translation or the different proteins can designed with different tags that can be used for binding different labeling reagents. In this way, fluorescence measurements, such as but not limited to FRET and TRET can be used to monitor protein-protein interactions in a phospholipids bilayer, including protein-protein interactions that occur within protein complexes having multiple proteins. In some aspects of the present invention, an NPS system can include a cell extract and nanoscale phospholipid bilayer discs in which the nanoscale phospholipid bilayer discs include components of the protein translocation machinery. Components of the protein translocation machinery can include Sec YEG proteins, can include mammalian counterparts, the protein translocation (pore-forming) proteins, SRP receptor, the ribosome receptor, etc., in order to facilitate membrane protein insertion. Other proteins such SecA, SecB, or FtsY (among others) might be exogenously added to the reaction. Chaperonins that aid in protein folding and membrane insertion can also be added.

Membrane protein components of the protein translocation machinery can be provide in pre-made PAPs, in which case the protein translocation proteins can be inserted through solubilization/dialysis methods of making PAPs, or can be inserted into PAPs using in vitro translation systems, as described herein.

The present invention also includes IVPS systems and methods that include PAP components, namely phospholipids and an apolipoprotein in soluble form, in which a MPOI is translated in the presence of PAP components and PAPs assemble in the reaction with the MPOI, such that the end result is a PAP with incorporated MPOI. Methods of making PAPs or “nanodiscs” is described in, for example, US Patent Application Publication No. 2005/0182243. The present invention includes providing solubilized PAP components, including apolipoproteins (such as but not limited to those disclosed herein and in US Patent Application Publication No. 2005/0182243) and phospholipids in an IVPS reaction, and providing a nucleic acid template that encodes a MPOI, such that the MPOI is translated in the presence of PAP components and becomes incorporated into a PAP in the context of the translation reaction. Assembly of PAPs can occur prior to the translation reaction, during translation, or following translation of an MPOI.

The methods of making PAPs by providing components in an IVPS system can be combined with other embodiments described herein, including, use of a tagged apolipoprotein, translation of MPOIs with PAP components on arrays or multiwell plates, translation of two or more MPOIs with PAP components, inclusion of components of the protein translocation machinery in the IVPS reaction mix that includes PAPs or PAP components, and translation of one or more components of the protein translocation machinery in the IVPS reaction mix that also includes PAPs or PAP components.

### Apolipoprotein-Membrane Protein Compositions

The present invention provides, in another embodiment, a composition that includes one or more membrane proteins associated with one or more apolipoproteins. Typically, the composition is a soluble, isolated complex of one or more apolipoproteins and one or more membrane proteins in an aqueous solution. The complex can include a lipid, such as a phospholipid. The complex of a membrane protein and an apolipoprotein can, in some embodiments, be substantially lipid-free. The membrane protein of the complex is typically synthesized using an in vitro protein synthesis system, as disclosed herein, typically in the presence of the apolipoprotein. A complex in illustrative examples of this embodiment of the invention can be free of detergents. The complex can also be a cell-free complex that includes an apolipoprotein, all or a portion of a membrane protein, typically at least the N-terminus portion, one or more ribosomes, and one or more RNA molecules, such as an RNA molecule encoding the membrane protein. The complex can include lipid or be substantially free of lipid. The complex can be an isolated complex. The complex can be optionally bound to a solid support via a nucleic acid template encoding either the apolipoprotein or the membrane protein, or via the apolipoprotein or membrane protein, either of which can optionally comprise a peptide tag.

The following examples are intended to illustrate but not limit the invention

### EXAMPLE 1

### In Vitro Expression of a Non-Membrane Protein in the Presence of Phospholipid-Apolipoprotein Particles

This example illustrates that the presence of nanodiscs in a prokaryotic in vitro translation system does not have a deleterious effect on the translation of non-membrane proteins.

In vitro protein synthesis reactions using plasmid DNA templates were assembled as follows: Standard 50 or 100 microliter EXPRESSWAY™ cell free expression system (Invitrogen, Carlsbad, Calif.) reactions were assembled and incubated at 37° C. essentially according to the manufacturer's instructions. The reactions included 600-800 micrograms of E coli extract made using an S30 buffer that contained 0.1% Triton-X 100 containing 2.5 micrograms per mL of Gam protein, 820U T7 Enzyme, 20U RNase Out, 0.5 microliters ³⁵S-Methionine, 1.25 mM amino acids, and 0.5-1 μg template DNA (either circular or linear) in 1× IVPS Buffer (58 mM Hepes, pH 7.6, 1.7 mM DTT, 1.2 mM ATP, 0.88 mM UTP, 0.88 mM CTP, 0.88 mM GTP, 34 micrograms per mL folinic acid, 30 mM actetyl phosphate, 230 mM potassium glutamate, 12 mM Magnesium Acetate, 80 mM NH₄OAc, 0.65 mM cAMP, 30 mM phosphoenolpyruvate, 2% polyethylene glycol). The template was the Cycle 3 GFP gene in the vector pCR2.1.

The reactions also included phospholipid-apolipoprotein nanoscale particles comprising a membrane scaffold protein and phospholipids, or “nanodiscs” as described in U.S. Patent Application Publication 2005/0182243 (U.S. patent application Ser. No. 11/033,489), herein incorporated by reference in its entirety, at a concentration (based on protein content) ranging from 1 micromolar to 40 micromolar. The PAPs were added from a stock solution that of 27 mg/mL PAP that were made of MSP1T2 scaffold protein (U.S. Patent Application Publication 2005/0182243) and DOPC. The reactions were performed in 1.5-2 ml microfuge tubes in an Eppendorf Thermomixer at either 30° C. or 37° C. with moderate shaking (1000-1400 rpm) for 2-6 hours. Reactions were fed one volume (with respect to initial reaction volume) of feeding solution 30 minutes after the start of the reaction. The feeding solution contained 57.5 mM HEPES-KOH pH 8.0, 230 mM Potassium Glutamate, 14 mM Magnesium Acetate, 80 mM Ammonium Acetate, 2 mM Calcium Chloride and 1.7 mM DTT. The feed also contained amino acids at 1.25 mM each (except for methionine, present at 1.5 mM), Glucose-6-Phosphate at 45 mM, NADH at 0.5 mM, 34 micrograms per milliliter folinic acid, and 0.65 mM cAMP. For radiolabeling of proteins, 2 microliters per 100 microliter reaction of ³⁵S-Methionine at a specific activity of 1175 ci/mmole was included in the reactions.

After the incubation was complete, in vitro protein synthesis reactions were spun briefly at 10,000×g and supernatant and pellet fractions were loaded separately on lanes of an SDS PAGE gel: 5 ul of each reaction supernatant was acetone precipitated, pelleted, and raised in 40 ul of 1× LDS buffer (Invitrogen, Carlsbad, Calif.) that included 1 mM DTT; 10 ul of this was loaded on 4-12% Bis/tris NuPAGE gels.

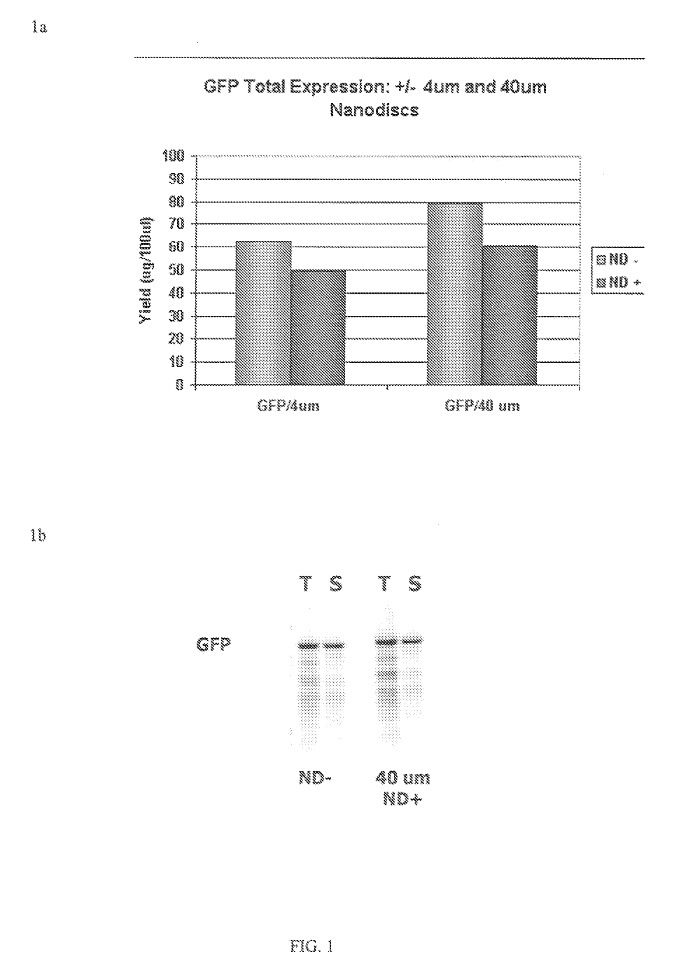

The total amount of GFP synthesized and the amount of soluble GFP was determined by autoradiography (FIG. 1). FIG. 1a provides a histogram based on autoradiography showing that including phospholipid-apolipoprotein nanoscale particles in the translation reaction at 4 micromolar and 40 micromolar does not have a substantially deleterious effect on the yield of a non-membrane protein. FIG. 1b shows an autoradiograph of total (lanes 1 and 3) and soluble (lanes 2 and 4) translation products synthesized in the presence (lanes 3 and 4) and absence (lanes 1 and 2) of 40 micromolar PAPs electrophoresed on a NuPAGE® Novex® 4-12% Bis-Tris gel (Invitrogen, Carlsbad, Calif.). The results indicate that the presence of phospholipid-apolipoprotein nanoscale particles in the translation reaction does not detectably increase the soluble fraction of a nonmembrane protein (GFP) synthesized in vitro.

### EXAMPLE 2

### In Vitro Synthesis of Membrane Proteins in the Presence of Nanodiscs

This example illustrates that the presence of phospholipids-apolipoprotein particles in an in vitro translation system enhances the yield of soluble synthesized membrane proteins of both prokaryotic and eukaryotic origin.

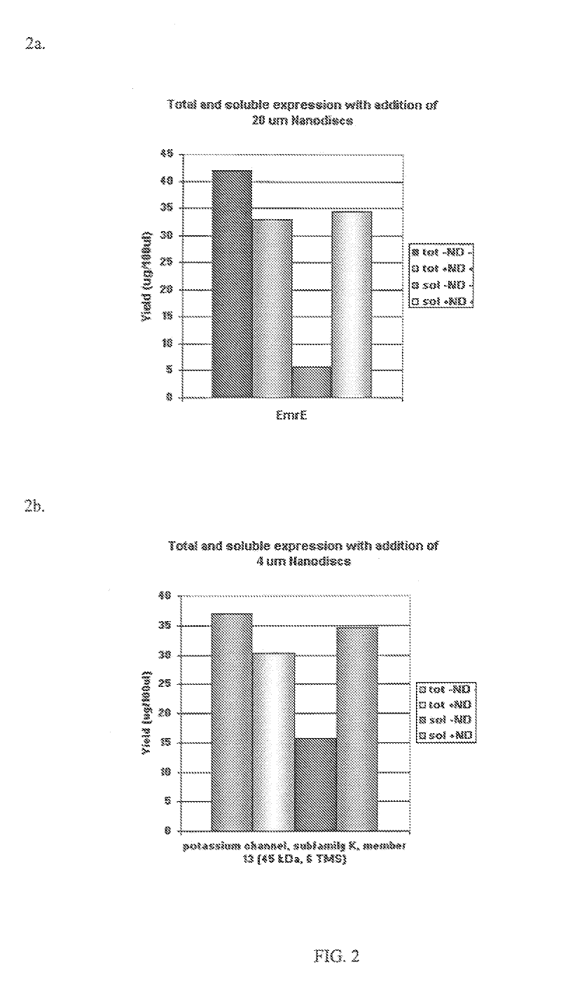

EmrE, a bacterial membrane protein (multidrug resistance protein), was translated using ³⁵S-Methionine in EXPRESSWAY™ cell free expression system (Invitrogen, Carlsbad, Calif.) reactions that included 20 micromolar PAPs. In vitro protein synthesis reactions were performed as described in Example 1. Total and soluble protein from the in vitro synthesis reactions were electrophoresed as described in Example 1. The results of autoradiography of a NuPAGE® Novex® 4-12% Bis-Tris gel (Invitrogen, Carlsbad, Calif.) on which the translation products were electrophoresed are depicted in histogram form in FIG. 2a. The presence of PAPs in the in vitro translation mix increased the yield of soluble EmrE protein by at least 5-fold.

Translation products were also electrophoresed on NativePAGE™ Novex® Bis-Tris 3-12% gels and autoradiographed to deteimine whether the synthesized proteins were present in complexes. EmrE protein translated in the absence of PAPs did not migrate into the gel but rather remained just at the bottom of the well, as it was presumably aggregated. EmrE protein synthesized in the presence of 20 or 25 micromolar PAPs entered the gel and migrated to a higher molecular weight range than did PAPs alone (not taken from an IVPS reaction). GFP, a soluble nonmembrane protein, migrated identically in a native gel whether it was synthesized in the presence or absence of PAPs, indicating it does not integrate into PAPs as EmrE, a membrane protein, does.

In addition, a mammalian membrane protein, the human potassium channel subfamily K, member 13 protein (Genbank accession no. NM 022054; gi 16306554, cDNA available from the Ultimate™ ORF clone collection, Invitrogen.com), a 45 kDa protein which has six transmembrane domains, was in vitro translated using EXPRESSWAY™ cell free expression system (Invitrogen, Carlsbad, Calif.) reactions as detailed in Example 1, in which the reactions included 4 micromolar PAPs. The reactions included 700 ng of the template, which was provided in the pEXP3 vector per 100 microliter reaction. Total and soluble protein from the in vitro synthesis reactions were electrophoresed as described in Example 1. The results of autoradiography of a NuPAGE® Novex® 4-12% Bis-Tris gel (Invitrogen, Carlsbad, Calif.) on which the translation products were electrophoresed are depicted in histogram form in FIG. 2b. In this case, the presence of PAPs increased the amount of soluble membrane protein by more than two-fold.

### EXAMPLE 3

### In Vitro Synthesized Membrane Proteins Co-Localize With2Phospholipid-Apolipoprotein Particles

This example demonstrates that the presence of phospholipid-apolipoprotein particles in an in vitro translation system results in the insertion of synthesized membrane proteins into PAPs.

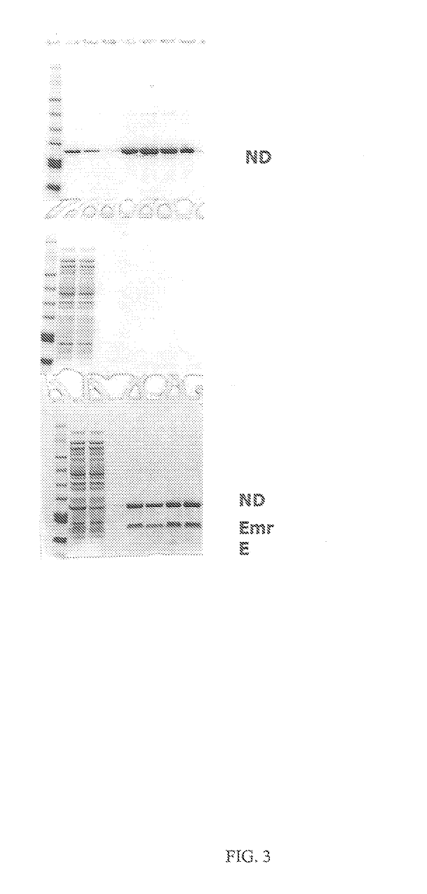

The apolipoprotein particle protein, or scaffold protein, MSP1T2, includes a his tag. Twenty micromolar PAPs made with the MSP1T2 his-tagged scaffold protein could be purified using a Ni-NTA resin (FIG. 3a, lanes 5-8 of a Coomassie-stained gel contain the column eluate fractions). As a control, EmrE protein was synthesized in a cell-free translation reaction containing ³⁵S-Methionine in the absence of PAPs, using EXPRESSWAY™ cell free expression system (Invitrogen, Carlsbad, Calif.) reactions as detailed in Example 1. No EmrE (which was not his-tagged) was purified on the Ni-NTA resin (FIG. 3b, lanes 5-8 contain the column eluate fractions). However, with addition of PAPs having a his-tagged engineered apolipoprotein protein to the reaction, however, EmrE (co-purifying with the phospholipids binding protein of the PAP) was purified on Ni-NTA resin (FIG. 3c, lanes 5-8 contain the column eluate fractions), thus demonstrating that EmrE was inserted into the PAPs having the purification tag.

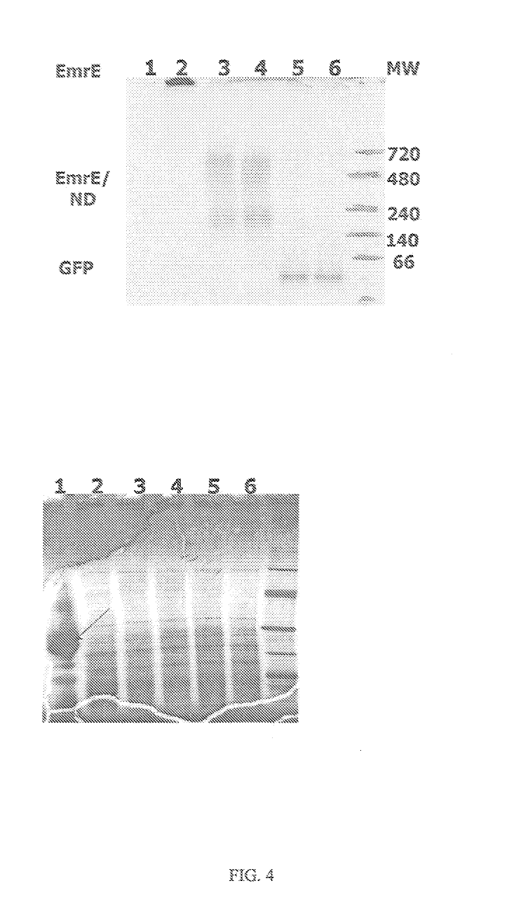

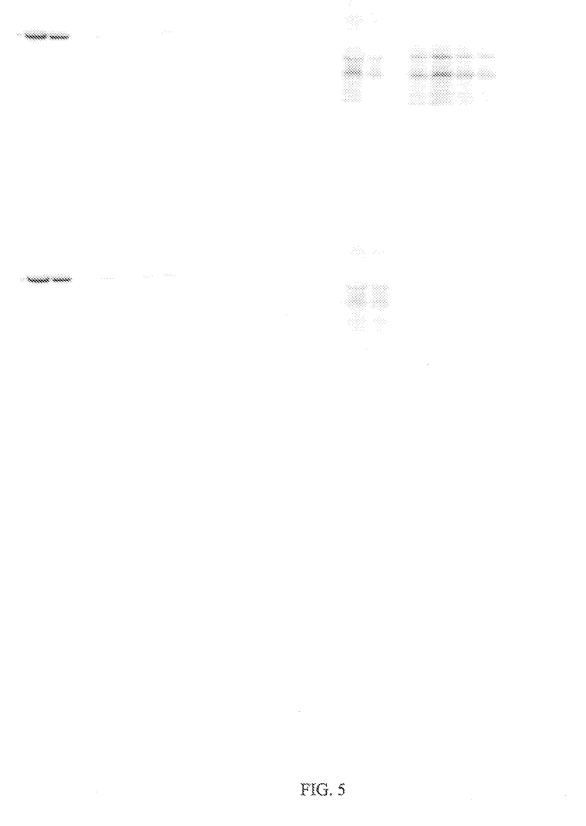

The result was verified by Native Blue gel analysis, in which radiolabeled bacterial membrane protein EmrE expressed without PAPs (about 0.3 micrograms of protein loaded) aggregated at the top of the gel (FIG. 5a, lane 2, autorad). When PAPs were added to the expression reaction, however, EmrE formed a complex (FIG. 4a, lanes 3, 4 autorad), which ran into the gel but migrated at a higher molecular weight than the PAPs alone (FIG. 4b, lane 1, Coomassie-stained gel). GFP, a non-membrane protein, ran at the same molecular weight with or without the addition of the PAPs to the translation system (FIG. 4a, lanes 5 and 6, respectively).

### EXAMPLE 4

Membrane Proteins Synthesized in Vitro with His-Tagged Nanodiscs can be Purified with Ni-NTA Resin

This example demonstrates that the presence of nanodiscs in an in vitro translation system allows for the purification of synthesized membrane proteins using tagged nanodiscs.

Genes for GFP and MscL, a bacterial mechanosensitive channel (17 kDa) membrane protein, were cloned the pEXP4 vector. Both genes contained a stop codon so the expressed proteins were not C-terminal His-tagged. PAPs (40 micromolar) that included his-tagged scaffold proteins were added to or omitted from the EXPRESSWAY™ cell free expression system (Invitrogen, Carlsbad, Calif.) reactions that included ³⁵S-Methionine and used one microgram of GFP and MscL templates. After incubation, the reactions were loaded onto Ni-NTA columns.

The results of column purification provided in FIG. 5 (L=load, FT=flowthrough, W=wash, E1-E4, elutions) show that GFP, which is not a membrane protein, cannot be purified using an Ni-NTA column, whether or not nanodiscs have been included in the translation reaction (FIGS. 5a and 5b). MscL, however, can be purified on an Ni-NTA column, but only when nanodiscs have been included in the translation reaction (FIGS. 5c and 5d). This shows that MscL inserts into PAPs.

### EXAMPLE 5

Membrane Proteins Synthesized in Vitro with Nanodiscs Associate with Nanodiscs and have Enhanced Solubility

This example demonstrates that the presence of phospholipid-apolipoprotein particles in an in vitro translation system results in the synthesis of membrane proteins having enhanced solubility that are inserted into PAPs enhanced solubility.

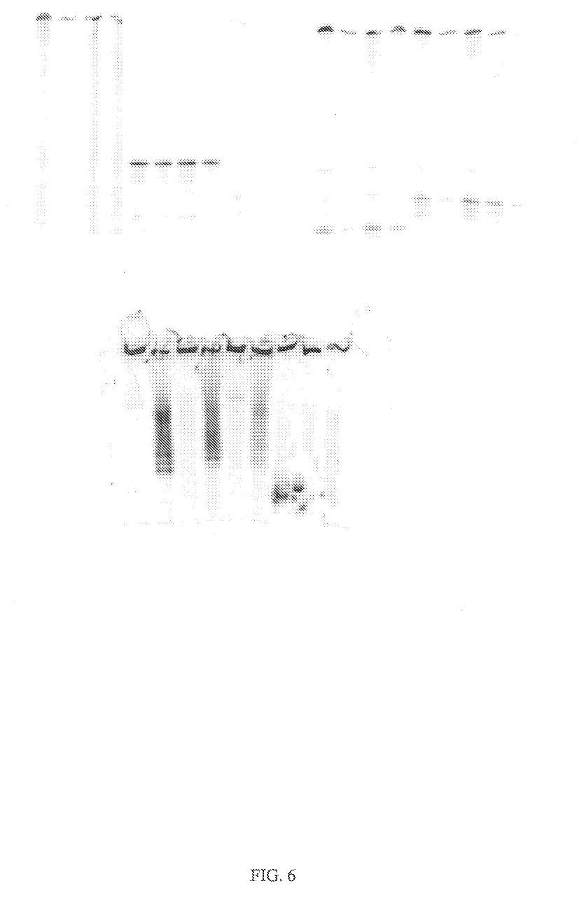

The bacterial membrane protein EmrE and mammalian ORFs “IOH 5384” (encoding human plasma membrane proteolipid (plasmolipin)) and “IOH22669” (encoding human adrenomedullin receptor (ADMR)) were expressed from the pEXP3 vector in EXPRESSWAY™ cell free expression system (Invitrogen, Carlsbad, Calif.) reactions that contained ³⁵S-Methionine. Running aliquots of the total protein and soluble fractions resulting from the in vitro synthesis reactions on NuPAGE® Novex® Bis-Tris gels (Invitrogen, Carlsbad, Calif.) shows that solubility of both the bacterial and mammalian membrane proteins is enhanced when PAPs are added to the in vitro synthesis reactions, but GFP solubility is not affected by the presence of PAPs in the in vitro synthesis reaction (FIGS. 6a and 6b).

The autoradiograph of a blue native gel shown in FIG. 6c shows that bacterial membrane protein EmrE, as well as mammalian ORFs IOH 5384 (encoding human plasma membrane proteolipid (plasmolipin)) and IOH22669 (encoding human adrenomedullin receptor (ADMR)), insert into PAPs. The radiolabeled EmrE, IOH 5384, and IOH22669 shift upward when PAPs are added to the reaction. GFP, a non-membrane protein, runs at the same molecular weight with or without the addition of the PAPs to in vitro synthesis reactions.

### EXAMPLE 6

LUMIO Detection of a Membrane Protein Inserted into PAPs

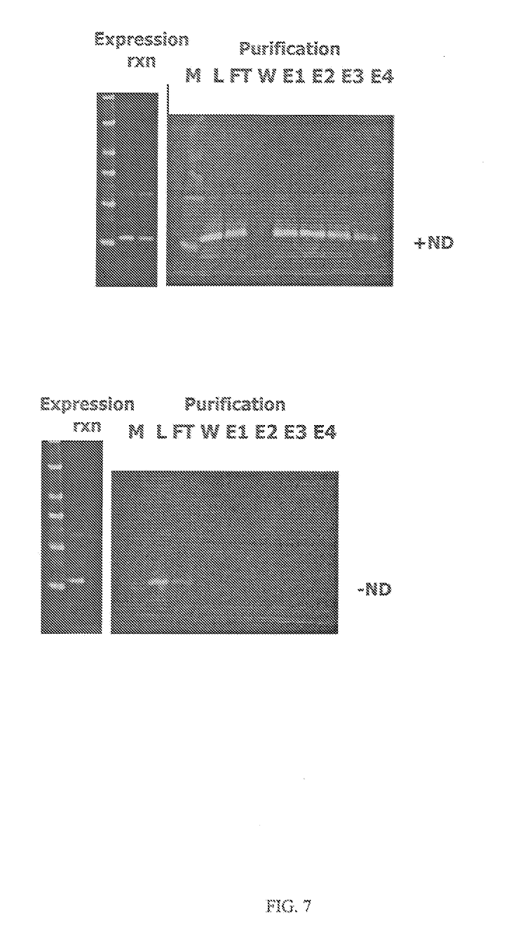

The gene for EmrE, a bacterial membrane protein, was cloned into an N-terminal vector containing a LUMIO™ tetracysteine motif tag (pEXP6, Invitrogen, Carlsbad, Calif.). The EmrE construct did not include His tag. PAPs (40 um) were added to or omitted from the EXPRESSWAY™ cell free expression system (Invitrogen, Carlsbad, Calif.) reactions, and at the end of the synthesis the reactions were loaded onto Ni-NTA columns (L=load, FT=flowthrough, W=wash, E1-E4, elutions). LUMIO™ detection reagent (Invitrogen, Carlsbad, Calif.) was added to samples before analysis on 4-12% NuPAGE® Bis-Tris gels according to manufacturer's instructions for the LUMIO™ Green in-gel detection kit (Invitrogen, Carlsbad, Calif.), and gels were imaged by a phosphorimager. In FIG. 7a, translation reactions that contained PAPs were analyzed. The LUMIO™ sequence (of the EmrE translation product) was detected in fractions eluted from the Ni-NTA column (purification using the His tag on scaffold protein of PAPs). In FIG. 7b, translation reactions that lacked PAPs were analyzed. The LUMIO™ sequence (of the EmrE translation product) was not detected in fractions eluted from the Ni-NTA column. Thus, membrane proteins can be synthesized in vitro in soluble form integrated into PAPs and efficiently purified using tags on the apolipoprotein of the PAP.

### EXAMPLE 7

### Eukaryotic in Vitro Protein Synthesis Reactions Containing Nanodiscs

Luciferase protein was expressed in a cell-free CHO cell extract that either did not contain PAPs, or contained from 0.1 to 19 micromolar PAPs. RNA was made from a pEXP4 vector that included the luciferase gene using mMessageMachine (AMBION). Six micrograms of RNA was used in translation reaction.

The CHO cell extract was made according to the following protocol:

### Determine Cell Count/Viability.

1. Collect the cells by gently centrifugation (10′×800-1000 rpm)
2. Add 4 mM DTT to buffer A
3. Wash the cells with 250 mL of buffer A (be very gentle; cells should not be fully resuspended)
4. Wash the cells with 250 mL of buffer A (simply add buffer; do not resuspend cells)
5. Resuspend the pellet in half pellet volume of buffer A (+1 mM PMSF)
6. Save an aliquot for cell count
7. Pass through French press at 100 psi
8. Save an aliquot for cell count
9. Determine cell count/viability in both aliquots. In the first aliquot most of the cells should be intact. In the second aliquot the cells should by <20% viable.
10. Centrifuge 15 min×14000 rpm (could be done in a microcentrifuge)
11. Collect the supernatant (and save the pellet at −80° C. for further use)
12. Add 1 mM CaCl₂, and 0.15 U/ul micrococcal nuclease.
13. Incubate for 5 min@RT
14. Stop the reaction with 2 mM EGTA
15. Aliquot in 50-80 ul samples, quickly freeze in liquid N₂ and store at −80° C.
16. Determine A₂₆₀ and A₂₈₀ of the supernatant ( 1/200 dilution). It should be >100 units.

### Buffer A

40 mM Hepes KOH pH 7.8
100 mM KOAc
4 mM Mg (OAc)2
4 mM DTT (add fresh)

Translations were performed using creatine kinase 5 mg/ml (0.5 ul), Buffer #2 Proteios wheat germ system (1.5 ul), RNaseOut (0.25 ul), Buffer #1 (0.85 ul), 35Smet (0.5 ul), and BHK extract (6 ul). The translation reactions were incubated at 33° C. for 1 hour. 2.5 ul of each translation reaction was used for luciferase analysis.

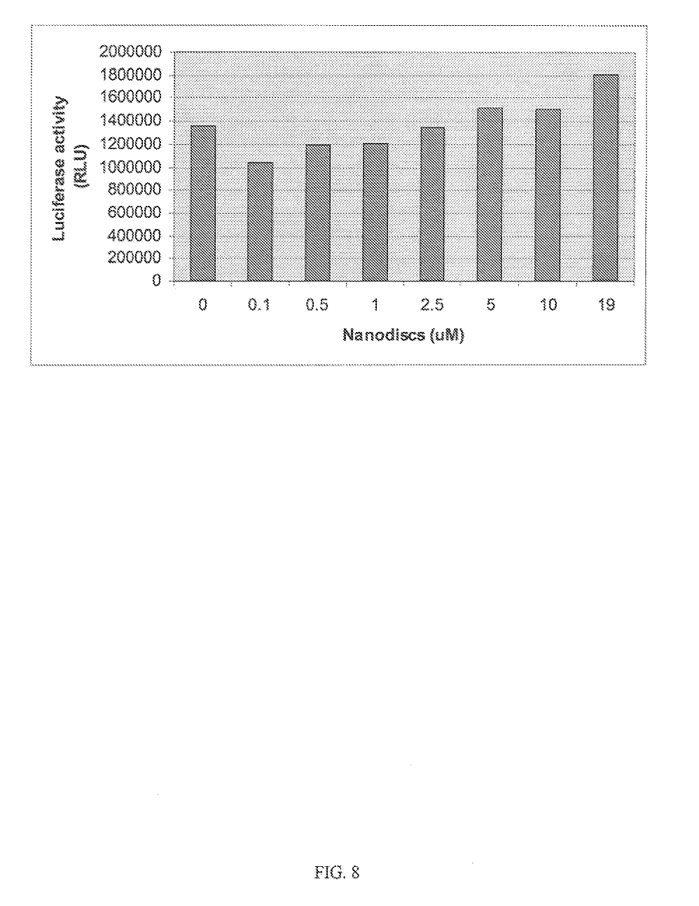

After the completion of the protein synthesis reactions, luciferase activity was detected. As shown in FIG. 8, the presence of PAPs did not have a detrimental effect of protein synthesis in the CHO cell extract.

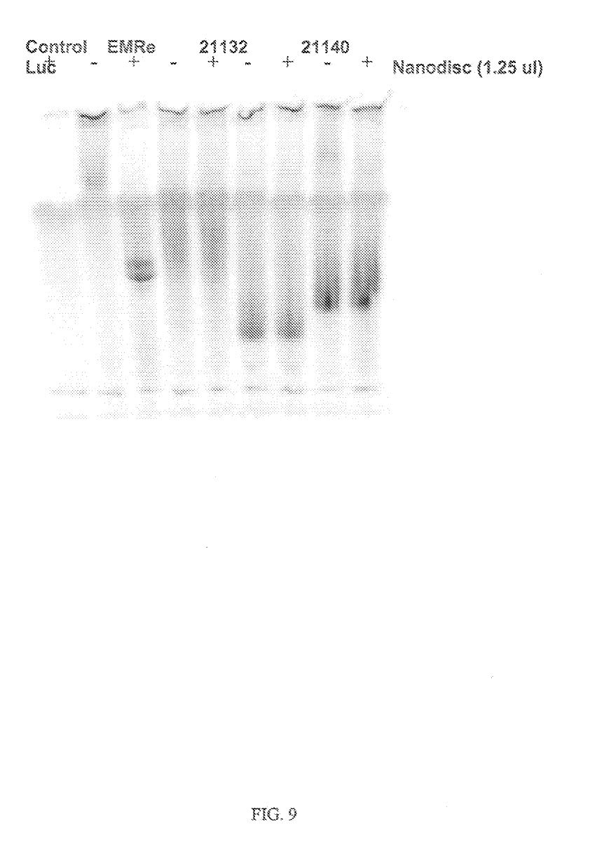

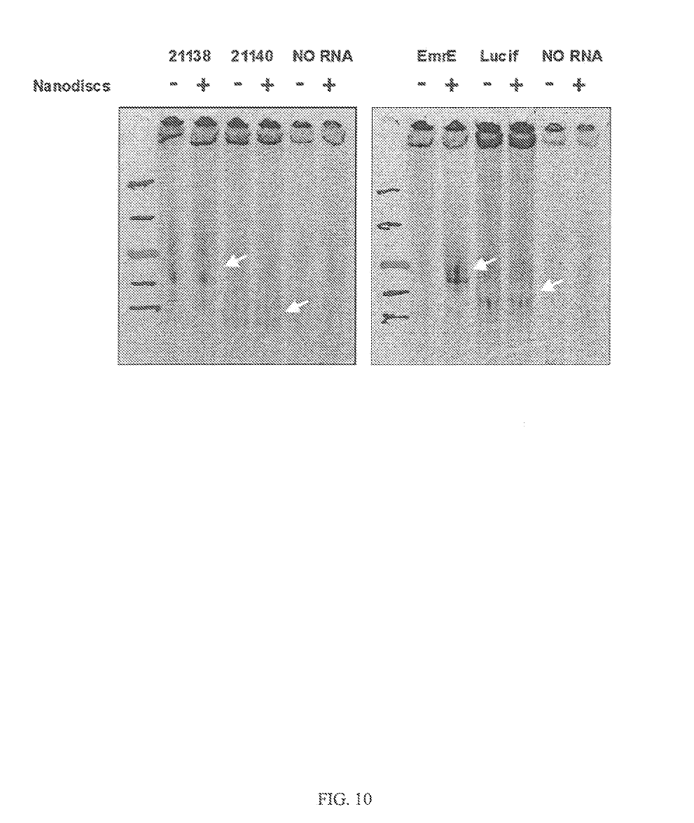

Bacterial membrane protein EmrE, human ORF 21132 (vesicle-associated calmodulin-binding protein), human ORF 21140 cyclin-dependent kinase 2 (CDK2), and luciferase were also translated in a coupled transcription-translation rabbit reticulocyte lysate system (Promega) that contained ³⁵S-Methionine according to the manufacturer's instructions. In one set of reactions, the synthesis system contained 1.25 microliters of 27 mg/mL PAPs. In duplicate reactions, the synthesis system did not have PAPs. The translation products were run on a Blue Native gel and autoradiographed. FIG. 9 shows that for membrane protein EmrE, increased solubility (radiolabeled protein products entering and migrating in the gel) was seen in the presence of PAPs. This was not the case for the non-membrane proteins human ORF 21132 (vesicle-associated calmodulin-binding protein), human ORF 21140 cyclin-dependent kinase 2 (CDK2), and luciferase. FIG. 10 shows the same result was obtained using a CHO cell extract.

### EXAMPLE 8

Enhanced Solubility of Membrane Proteins Co-Expressed with an Apolipoprotein in In Vitro Synthesis Reactions

This example illustrates that the presence of an apolipoprotein construct in an in vitro translation system in the absence of PAPs, promotes the synthesis of membrane proteins in soluble form.

In separate experiments, a bacterial membrane protein and a mammalian membrane protein were transcribed and translated from plasmid constructs in cell-free synthesis systems. In one set of experiments, the protein of interest (POI) construct contained a gene encoding bacterial membrane protein EmrE cloned in vector pEXP5-NT. In another set of experiments, the protein of interest or “first” construct contained a gene encoding human membrane protein GABA A receptor (Invitrogen ULTIMATE™ ORF collection clone IOH10885, Genbank accession no. BC 022449, gi 18490266;) cloned in vector pEXP5-NT. In both experiments, a construct encoding human Apolipoprotein A1 (Invitrogen ULTIMATE™ ORF collection clone IOH7318, Genbank accession no. NM 00839, gi 4557320, pEXP3-Apo1, was also included in some of the in vitro synthesis reactions, so that Apo A1 was translated in the same reaction as the membrane protein of interest.

In vitro protein synthesis reactions using plasmid DNA templates were assembled as follows: Standard 100 microliter EXPRESSWAY™ reactions were assembled and incubated at 37° C. essentially according to the manufacturer's instructions (Invitrogen Corp, Carlsbad, Calif.). 1 ug of DNA construct was added to each of the reactions. The reactions were performed in 1.5-2 ml microfuge tubes in an Eppendorf Thermomixer at either 30° C. or 37° C. with moderate shaking (1000-1400 rpm) for 2-6 hours. Reactions were fed one volume (with respect to initial reaction volume) of feeding solution 30 minutes after the start of the reaction, as per manufacturer's instructions (Invitrogen Expressway manual, Invitrogen Corp., Carlsbad, Calif.). For radiolabeling of proteins, ³⁵S-Methionine was included in the reactions.

For each membrane protein of interest, reactions were performed with and without pEXP3-Apo1. In addition, each set of reactions was performed with and without added phospholipids, either 100 micrograms per milliliter of DMPC, in the case of EmrE translation reactions, or 100 micrograms per milliliter of DPPC, in the case of GABA A receptor translation reactions.

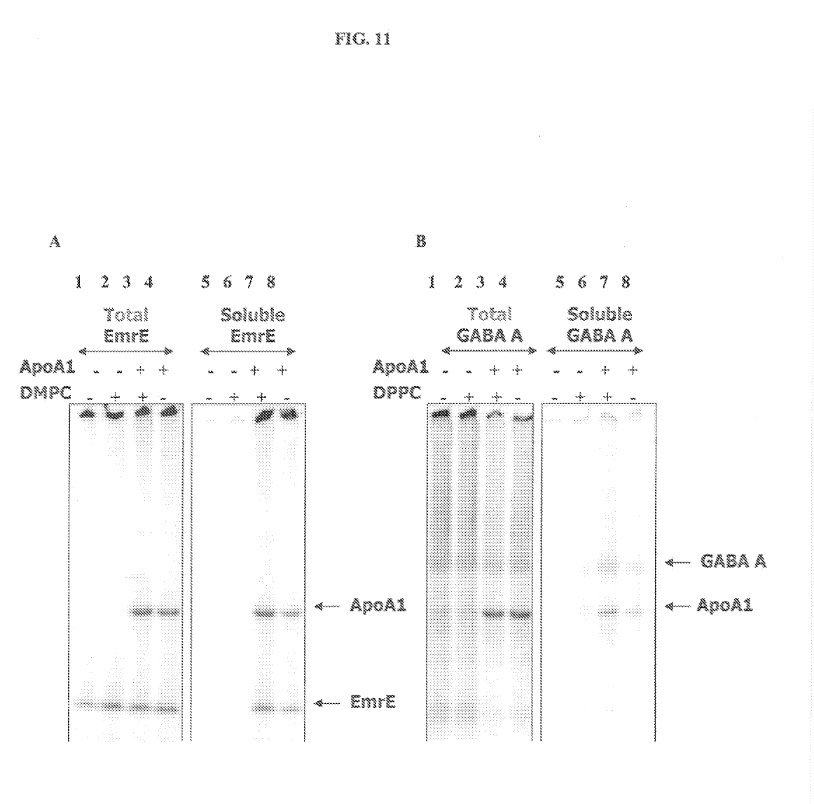

After the incubation was complete, in vitro protein synthesis reactions were spun briefly at 10,000×g and supernatant and pellet fractions were loaded separately on lanes of an SDS PAGE gel: 5 ul of each reaction supernatant was acetone precipitated, pelleted, and raised in 40 ul of 1× LDS buffer (Invitrogen) that included 1 mM DTT; 10 ul of this was loaded on 4-12% Bis-Tris NuPAGE® gels. (FIG. 11).

The results show that the presence of the Apo A1 construct in the translation reactions greatly improves the yield of soluble EmrE (lanes 7 & 8, FIG. 11a) compared to translation in the absence of the Apo A1 construct (lanes 5 & 6). Apo A1 also greatly improves the soluble yield of GABA A (lanes 7 & 8, FIG. 11b) when compared with soluble yield in the absence of the Apo A1 construct (lanes 5 & 6). The autoradiographs also clearly show that Apolipoprotein A1 itself is translated in soluble form (Lanes 3 and 4 of FIGS. 11a and 11b).

### EXAMPLE 9

### In Vitro Synthesis of Membrane Proteins in the Presence of Apolipoprotein

This example illustrates that the presence of an apolipoprotein in an in vitro translation system enhances the yield of soluble synthesized membrane proteins.

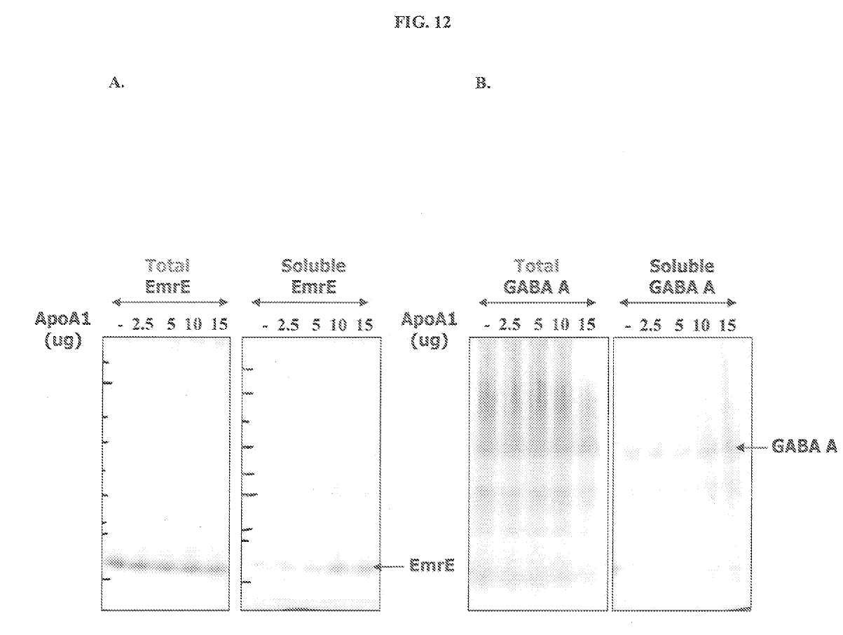

EmrE, a bacterial membrane protein, was translated using ³⁵S-Methionine in an EXPRESSWAY™ in vitro synthesis system (Invitrogen Corp., Carlsbad, Calif.) as described in the previous example that also included from 2.5 to 15 micrograms of Apo A1 protein. Total and soluble protein from the in vitro synthesis were electrophoresed on gels and subjected to autoradiography. The results (FIG. 12a) show that increasing the amount of Apo A1 protein in the in vitro synthesis reaction greatly increases the amount of solubilized membrane protein made.

In addition, a mammalian membrane protein, the human GABA A receptor protein, was in vitro translated in a system that included from 2.5 to 15 micrograms of Apo A1 protein. In this case as well, the presence of Apo A1 protein in the translation system greatly increased the amount of soluble membrane protein (FIG. 12b).

### EXAMPLE 10

Apolipoproteins of in Vitro Synthesis System Associate with Translated Membrane Proteins

This example demonstrates that the presence of Apo A1 protein in an in vitro translation system in which a membrane protein is synthesized results in the synthesis of soluble membrane protein, and that Apo A1 associates with the translated protein. The example also demonstrates that solubility of a membrane protein that normally requires the presence of detergent can be maintained in the absence of detergent when an apolipoprotein is present.

Expressway in vitro translation reactions were performed in a total volume of 100 microliters. The nucleic acid template was the EmrE gene cloned in pEXP5-NT (Invitrogen, Carlsbad, Calif.) which encodes a his tag positioned N-terminal to, and in frame with, the insert. Apo A1 purified from human plasma was included in the in vitro synthesis reaction. After perfoming in vitro synthesis reactions according to manufacturer's instructions, the his-tagged in vitro synthesized EmrE was purified on an Ni-NTA column by gravity flow using 20 mM Tris, pH 7.5, 200 mM NaCl. Dodecyl maltoside detergent, usually included in buffers to maintain EmrE solubility, was omitted.

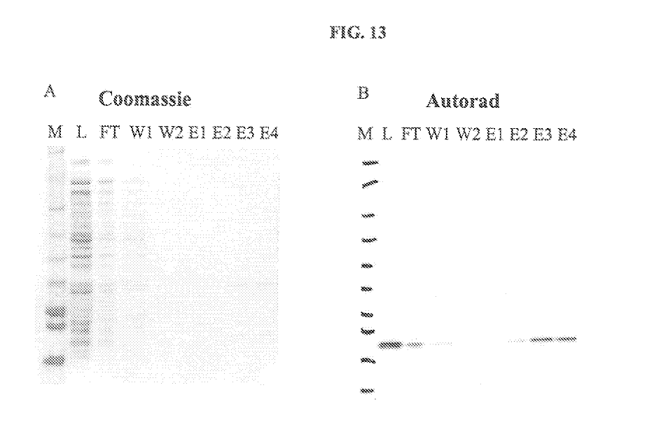

The EmrE protein was eluted using 1 M imidazole in the same Tris-NaCl buffer. The Coomassie gel shown in FIG. 13a shows that the untagged Apo A1 protein (26 kDa) co-purified with the His-tagged EmrE. (M indicates protein molecular weight markers, L indicates the loaded fraction, FT indicates flow through, W1 and W2 are successive column washes, and E1-E4 are successive elution fractions. The autoradiograph (FIG. 13b) confirms the EmrE protein eluted in the same fractions as purified Apo A1 protein, demonstrating a physical association between the proteins.

1. A method of synthesizing a membrane protein, comprising:
adding a nucleic acid template to an in vitro protein synthesis system comprising a cell extract and a phospholipid-apolipoprotein A-I particle; and
incubating the in vitro protein synthesis system with the nucleic acid template to synthesize the membrane protein in soluble form.

2. The method of claim 1, wherein said membrane protein is a transmembrane protein, an embedded membrane protein, or a peripheral membrane protein.

3. The method of claim 1, wherein said membrane protein is synthesized in soluble form.

4. The method of claim 1, wherein said cell extract is a prokaryotic cell extract.

5. The method of claim 4, wherein said cell extract is an E. coli cell extract.

6. The method of claim 1, wherein said cell extract is a eukaryotic cell extract.

7. The method of claim 6, wherein said cell extract is a wheat germ extract, a Drosophila embryo extract, a rabbit reticulocyte extract, a scallop extract, a mouse brain extract, a chick brain extract, or an extract of cultured cells.

8. The method of claim 1, wherein said nucleic acid template is an RNA template.

9. The method of claim 1, wherein said nucleic acid template is a DNA template.

10. The method of claim 1, wherein the apolipoprotein is present in a phospholipid-apolipoprotein particle.

11. The method of claim 1, wherein said apolipoprotein A-I present in the phospholipid-apolipoprotein A-I particle is a naturally-occurring apolipoprotein A-I, a variant of a naturally-occurring apolipoprotein A-I, or an engineered apolipoprotein A-I.

12. The method of claim 1, wherein said apolipoprotein is Alipoprotein A1 or a variant thereof.

13. The method of claim 1, wherein the apolipoprotein A-I present in the phospholipid-apolipoprotein A-I particle is an engineered apolipoprotein A-I that comprises at least one amphipathic helical domain.

14. The method of claim 1, wherein the apolipoprotein A-I present in the phospholipid-apolipoprotein A-I particle that comprises at least one amino acid sequence tag.

15. The method of claim 14, wherein said at least one amino acid sequence tag is a His tag.

16. The method of claim 14, further comprising purifying the membrane protein using the sequence tag of the apolipoprotein A-I.

17. An in vitro protein synthesis system comprising:
a cell extract;
at least one energy source; and
an apolipoprotein.

18. The in vitro protein synthesis system of claim 17, wherein said cell extract is a prokaryotic cell extract.

19. The in vitro protein synthesis system of claim 17, wherein said in vitro synthesis system comprises an E. coli cell extrqact.

20. The in vitro protein synthesis system of claim 17, wherein said cell extract is a eukaryotic cell extract.
